SEQUENCE LISTING (11 sequences)

SEQ ID NO: 1 - DNA, 29 bp, Artificial Sequence
  Primer NT-3 Fwd
actacgcgtatgtccatcttgttttatgt

SEQ ID NO: 2 - DNA, 27 bp, Artificial Sequence
  Primer NT-3 Rev
cttgaattctcatgttcttccaatttt

SEQ ID NO: 3 - DNA, 29 bp, Artificial Sequence
  Primer IL-10 Fwd
actggatccatgcctggctcagcactgct

SEQ ID NO: 4 - DNA, 31 bp, Artificial Sequence
  Primer IL-10 Rev
cttgcggccgcttagcttttcattttgatca

SEQ ID NO: 5 - DNA, 26 bp, Artificial Sequence
  Primer LINGO-1-Fc Fwd
actggatccccatgtacaggatgcaa

SEQ ID NO: 6 - DNA, 31 bp, Artificial Sequence
  Primer LINGO-1-Fc Rev
cttgcggcctatttacccggagacagggaga

SEQ ID NO: 7 - DNA, 29 bp, Artificial Sequence
  Primer copGFP Fwd
actacgcgtatggagagcgacgagagcgg

SEQ ID NO: 8 - DNA, 29 bp, Artificial Sequence
  Primer copGFP Rev
cttggatccttagcgagatccggtggagc

SEQ ID NO: 9 - DNA, 777 bp, Mus musculus
atgtccatcttgttttatgtgatatttcttgcttatctccgtggcatccaaggcaacagcatggatcaaaggagtttgccggaagactctctcaattccctcatcatcaagctgatccaggcggatatcttgaaaaacaagctttccaaacagatggtggatgttaaggaaaattaccagagcaccctgcccaaagcagaggcacccagggaaccagagcagggagaggccaccaggtcagagttccagccaatgattgcaacggacacagagctactacggcaacagagacgctacaattcgccccgggtcctgctgagtgacagcacccctttggagccccctcccttatacctaatggaggattatgtgggcaacccggtggtagccaatagaacctcaccacggaggaaacgctatgcagaacataagagtcaccgaggagagtactcagtgtgtgacagtgagagcctgtgggtgaccgacaagtcctcagccattgacattcggggacaccaggtcacagtgctgggggagatcaaaaccggtaactctcctgtgaaacaatatttttatgaaacgagatgtaaagaagccaggccggtcaaaaacggttgcagggggattgatgacaaacactggaactctcagtgcaaaacttcgcaaacctatgtccgagcactgacttcagaaaacaacaaactcgtaggctggcgctggatacgaatagacacttcctgtgtgtgtgccttgtcgagaaaaattggaagaacatga

SEQ ID NO: 10 - DNA, 537 bp, Mus musculus
atgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 11 - DNA, 1863 bp, Mus musculus
atgcaggtgagcgagaggatgctggcagggggtatgagaagcatgcccagccccctcctggcctgctggcagcccatcctcctgctggtactgggctcagtgctgtcaggctctgctacaggctgcccgccccgctgcgagtgctcagcgcaggaccgagccgtgctctgccaccgcaaacgctttgtggcggtgcccgagggcatccccaccgagactcgcctgctggacctgggcaaaaaccgcatcaagacactcaaccaggacgagtttgccagcttcccacacctggaggagctagaactcaatgaaaacatcgtgagcgccgtggagccaggcgccttcaacaacctcttcaacctgaggactctggggctgcgcagcaaccgcctgaagcttatcccgctgggcgtcttcaccggcctcagcaacttgaccaagctggacatcagtgagaacaagatcgtcatcctgctagactacatgttccaagacctatacaacctcaagtcgctggaggtcggcgacaacgacctcgtctacatctcccatcgagccttcagcggcctcaacagcctggaacagctgacgctggagaaatgcaatctgacctccatccccacggaggcgctctcccacctgcacggcctcatcgtcctgcggctacgacatctcaacatcaatgccatcagggactactccttcaagaggctgtaccgacttaaggtcttagagatctcccactggccctacctggacaccatgacccccaactgcctctacggcctcaacctgacatccctatccatcacgcactgcaacctgacagccgtgccctatctggcagtgcgtcacctggtctatctccgtttcctcaacctttcctacaacccaatcggtacaatcgagggctccatgctgcatgagctgctgcggttgcaggagatccagctggtgggcgggcagctggccgtggtggagccctatgcctttcgtgggctcaactacctgcgtgtgctcaatgtctctggcaaccagctgaccaccctggaggagtcagccttccattcggtgggcaacctggagacgctcatcctggactccaacccactggcctgtgactgccggctgctgtgggtgttccggcgccgctggcggctcaacttcaacaggcagcagcccacctgcgccacacctgagttcgtccagggcaaagagttcaaggactttccggatgtactcctacccaactacttcacctgccgccgggcccacatccgggaccgcaaggcacagcaggtgtttgtagatgagggccacacggtgcagtttgtatgccgggcagatggcgaccctccaccagctatcctttggctctcaccccgcaagcacttggtctcggccaagagcaatgggcggctcacagtcttccctgatggcacgctggaggtgcgctacgcccaggtacaggacaacggcacgtacctgtgcatcgcagccaatgctggcggcaacgactccatgcccgcccacttgcatgtgcgcagctactcgcctgactggccccatcaacccaacaagaccttcgccttcatctccaaccagccaggcgagggagaggccaacagcacccgcgccactgtgcctttccccttcgacatcaagacgctcattatcgccaccaccatgggcttcatctccttcctgggcgttgtcctattctgcctggtgctgctgtttctatggagccggggcaaaggcaacacaaagcacaacatcgaaattgagtatgtgccccggaaatcggacgcaggcatcagctcagctgatgcaccccgcaagttcaacatgaagatgatatga

(19) United States
(12) Patent Application Publication
(10) Pub. No.: US 2020/0197487 A1
(43) Pub. Date: Jun. 25, 2020

(54) A TRIPLE-EFFECT COCKTAIL PRODUCED BY NEURAL STEM CELLS AS A NOVEL NEUROREPAIR THERAPY FOR CHRONIC STAGE CNS AUTOIMMUNITY

(71) Applicant: Thomas Jefferson University, Philadelphia, PA (US)

(72) Inventors: Guang-Xian Zhang, Wynnewood, PA (US); Yaping Yan, Philadelphia, PA (US); Xing Li, Haddonfield, NJ (US)

(73) Assignee: Thomas Jefferson University, Philadephia, PA (US)

(21) Appl. No.: 16/609,882

(22) PCT Filed: May 7, 2018
(86) PCT No.: PCT/US18/31402

§ 371 Date: Oct. 31, 2019

Related U.S. Application Data
(60) Provisional application No. 62/502,206, filed on May 5, 2017.

(51) Int. Cl. A61K 38/18; A61K 35/30; A61K 38/17; A61K 38/20; C12N 5/0793; C12N 5/0797; A61P 25/28

(52) U.S. Cl. CPC A61K 38/185; A61K 35/30; A61K 38/177; A61P 25/28; C12N 5/0619; C12N 5/0623; A61K 38/2066

(57) ABSTRACT

Treatment of chronic neurodegenerative diseases such as multiple sclerosis (MS) remains a major challenge. Here we genetically engineer neural stem cells (NSCs) to produce a triply therapeutic cocktail comprising IL-10, NT-3, and LINGO-1-Fc, thus simultaneously targeting all mechanisms underlie chronicity of MS in the central nervous system (CNS): persistent inflammation, loss of trophic support for oligodendrocytes and neurons, and accumulation of neuroregeneration inhibitors. After transplantation, NSCs migrated into the CNS inflamed foci and delivered these therapeutic molecules in situ. NSCs transduced with one, two, or none of these molecules had no or limited effect when injected at the chronic stage of experimental autoimmune encephalomyelitis; cocktail -producing NSCs, in contrast, mediated the most effective recovery through inducing M2 macrophages/microglia, reducing astrogliosis, and promoting axonal integrity and endogenous oligodendrocyte/neuron differentiation. These engineered NSCs simultaneously target major mechanisms underlying chronicity of MS and EAE, thus representing a novel and potentially effective therapy for the chronic stage of MS, for which there is currently no treatment available.

### PRIORITY CLAIM

This application claims priority to U.S. Provisional Patent Application No. 62/502,206 filed May 5, 2017, which is hereby incorporated by reference in its entirety.

### GOVERNMENT FUNDING

This invention was made with government support under Grant Nos. NS075260 and NS069954 awarded by the National Institutes of Health. The government has certain rights in the invention.

### SEQUENCE LISTING

The instant application contains a Sequence Listing which has been filed electronically in ASCII format and is hereby incorporated by reference in its entirety. Said ASCII copy, created on May 1, 2018, is named 6107_177PCT_Sequence_Listing_SL.txt and is 6,114 bytes in size.

### FIELD OF INVENTION

The present application is related to a novel system on the chronic stage of EAE using adult NSCs that were engineered to simultaneously produce a therapeutic cocktail (cocktail-NSCs) containing IL-10, neurotrophin 3 (NT-3), and soluble LINGO-1 protein (LINGO-1-Fc), under the control of the Tet-on system.

### BACKGROUND OF INVENTION

Multiple sclerosis (MS) and its animal model, experimental autoimmune encephalomyelitis (EAE), are inflammatory demyelinating disorders of the central nervous system (CNS) [1, 2]. MS is thought to begin when peripherally activated myelin-reactive T cells infiltrate the CNS, followed by other immune cells, including monocytes, B cells, and neutrophils. In the CNS, these cells activate microglia and astrocytes and produce IFN-γ, IL-17, GM-CSF and IL-23, which promote inflammatory responses [3], while immunoregulatory cytokines like IL-4, IL-10 and IL-27 may be protective [4-6]. Three major mechanisms contribute to the chronic progression in MS: 1) persistent CNS inflammation [7]; 2) loss of trophic support for both oligodendrocytes and neurons [8]; and 3) accumulation of CNS regeneration inhibitors such as Nogo-A, myelin-associated glycoprotein (MAG) and oligodendrocyte myelin glycoprotein (OMgp) [9]. Persistent CNS inflammation results in continuous CNS tissue damage, i.e., demyelination, axonal degeneration and neuronal dysfunction [10], while myelin proteins from the damaged tissue, in turn, perpetuate chronic inflammation and the production of neuroregeneration inhibitors in the disease foci [8, 9]. This vicious cycle results in failure of spontaneous remyelination, axonal loss, and disease progression [11].

Current MS therapies mainly target dysfunctional immune response, one of the above-mentioned three pathogenic mechanisms; they are thus only partially effective in mitigating disease progression and are less effective at the chronic stage [12]. These immunomodulatory agents have little or no effect on the lack of neurotrophic factors and/or the accumulation of neuroregeneration inhibitors in disease foci. On the other hand, delivery of a neurotrophic factor may primarily modulate myelination in the CNS and promote oligodendrocyte precursor (OPC) proliferation, survival, and differentiation [13], while its effects will likely be diminished by inflammation. Blocking neuroregeneration inhibitors in the CNS partially suppresses EAE, while having no impact on CNS inflammation [14]. An MS therapy that targets these three pathogenic mechanisms at the same time would therefore be most beneficial.

In view of the beneficial effects of neural stem cells (NSCs) in EAE treatment, including their capacity for neuroprotection, immunomodulation, trophic function, and possibly neuro-repopulation, they appear to be an excellent candidate for cell-based therapy. Indeed, NSCs, even without any modification, suppress EAE development [15-20]. However, NSC therapy in EAE has resulted in only marginal improvement in clinical score for acute EAE [15, 16, 21, 22], while their effect on the chronic stage of disease has not been studied. Our pilot experiments showed that unmodified NSCs failed to induce recovery from EAE when injected at day 60 post immunization (p.i.) (data not shown). The main reasons for this lack of therapeutic effect may be the poor survival and low differentiation potential of transplanted NSCs in CNS lesions, and their suboptimal immunoregulatory capacity [6, 16, 22]. An approach that significantly enhances therapeutic capacity of NSCs in chronic stage of EAE is, therefore, crucial. Further, given that transplanted NSCs migrate only into the CNS inflamed foci [23], these cells have been a useful vehicle to deliver therapeutic molecules to the disease foci for EAE treatment [16, 21].

### SUMMARY OF INVENTION

The present invention is directed to a novel system on the chronic stage of Multiple Sclerosis using adult NSCs that were engineered to simultaneously produce a therapeutic cocktail (cocktail-NSCs) containing IL-10, an effective immunoregulatory cytokine; neurotrophin 3 (NT-3), a potent neurotrophic factor; and soluble LINGO-1 protein (LINGO-1-Fc), an antagonist of LINGO-1, a key part of the common receptor complex for neuroregeneration inhibitors [24], under the control of the Tet-on system.

In certain embodiments the invention is directed to a therapeutic comprising NSCs engineered to simultaneously produce a cocktail containing IL-10, neurotrophin3, and soluble LINGO-1 protein, which can be administered to a patient in an effective dose for treatment of Multiple Sclerosis.

In certain embodiments the invention is directed towards a method of treating multiple sclerosis comprising administering to a patient an effective dose of an engineered NSC producing a cocktail containing IL-10, neurotrophin3, and soluble LINGO-1 protein.

In certain embodiments the invention is directed towards a method of transgenically modifying an NSC cell comprising: inserting NT-3, IL-10, and LINGO-1-Fc genes individually into separate vectors and transduced them into cells sequentially, wherein said transgenic cell is thereafter capable of producing IL-10, neutrophin 3 and LINGO-1 protein.

A therapeutic for treatment of multiple sclerosis comprising neural stem cells (NSCs) engineered with a vector to insert cDNA corresponding to neurotrophin 3 (NT-3), IL-10, and LINGO-1, wherein said NSC to simultaneously produce a cocktail containing IL-10, neurotrophin3, and soluble LINGO-1 protein. Preferably, the therapeutic, wherein said NSC comprises a lenti virus vector inserting the cDNA sequence corresponding to NT-3, IL-10, and LINGO-1. Preferably, the therapeutic, wherein said NSC comprises SEQ ID Nos. 9, 10, and 11. Preferably, the therapeutic, when administered is suitable to inhibit pro-inflammatory M1 phenotype of microglia and switch it to M2 phenotype.

A preferred embodiment is directed towards a method of treating multiple sclerosis comprising administering to a patient an effective dose of an engineered NSC producing a cocktail containing IL-10, neurotrophin3, and soluble LINGO-1 protein. The method, wherein said NSC comprises a vector inserting a sequence corresponding to NT-3, IL-10, and LINGO-1. The method, wherein said NSC comprises cDNA comprising SEQ ID Nos. 9, 10, and 11. Preferably, the cDNA comprises 70% or 80%, or 90% or 99% homology to SEQ ID Nos. 9, 10, and 11. The method, wherein said method inhibits pro-inflammatory M1 phenotype of microglia and switches it to M2 phenotype.

A preferred embodiment is directed towards a method of transgenically modifying an NSC cell comprising: inserting NT-3, IL-10, and LINGO-1-Fc genes individually into separate vectors and transduced them into cells sequentially, wherein said transgenic cell is thereafter comprises genes corresponding to NT-3, IL-10, and LINGO-1, and wherein said transgenically modified NSC cells is capable of expressing NT-3, IL-10, and LINGO-1.

In preferred embodiments, the method of transgenically modifying the NSC cells further comprising generating a vector comprising NT-3 cDNA having primers corresponding to SEQ ID Nos 1, and 2. In preferred embodiments, wherein the NT-3 cDNA comprises SEQ ID No. 9, or at least 70% or more homology to SEQ ID NO. 9. In preferred embodiments, the method comprising generating a vector comprising IL-10 cDNA having primers corresponding to SEQ ID Nos. 3, and 4. In preferred embodiments, wherein the IL-10 cDNA comprises SEQ ID No. 10, or a sequence having at least 70% or more homology to SEE ID No. 10. In preferred embodiments, the method comprising generating a vector comprising LINGO-1-Fc cDNA having primers corresponding to SEQ ID Nos. 5, and 6. In preferred embodiments, wherein the LINGO-1-Fc cDNA comprises SEQ ID No. 11, or a cDNA having at least 70% or more homology to SEQ ID No. 11.

In certain embodiments, the methods comprise generating a vector comprising copGFP having primers corresponding to SEQ ID Nos. 7, and 8, wherein the GFP enables confirmation of the success of the insertion of the lentivirus.

In preferred embodiments, the method of transgenically modifying an NSC cell, comprising sequentially inserting each of NT-3, IL-10, and LINGO-1-Fc into multiple cloning sites of at least one vector. In certain embodiments, wherein said at least one vector is pLVX-EF1a-Tet3G. In certain embodiments, wherein said at least one vector is pLVS-TRE3G-IRES. In further embodiments, wherein said at least one vector is a first and second vector comprising pLVX-EF1a and pLVX-TRE3G-IRES. In certain embodiments, wherein said first vector comprises SEQ ID Nos. 9 and 10, and said second vector comprises SEQ ID no. 11.

A preferred embodiment is directed towards a method of producing a transgenic neural stem cell inducibly expressing multiple therapeutic genes comprising: cloning and inserting the NT-3 (SEQ ID No. 9) and IL-10 (SEQ ID No. 10) cDNA into multiple cloning sites (MCSs) of the pLVX-TRE3G-IRES-puroycin vector; said insertions are completed with primers comprising SEQ ID Nos. 1-4; replacing the puromycin gene with a hygromycin gene in the pLVX-TRE3G-IRES-puro vector, and inserting LINGO-1-Fc (SEQ ID No. 11) and into pLVX-TRE3G-IRES-puro vector after replacement of the puromycin gene with the hygromycin gene. A preferred embodiment, wherein copGFP cDNA is also inserted into the MCS of pLVX-TRE3G-IRES-puro for insertion into the cell. In a preferred embodiment, wherein the vectors are produced with a lentivirus. In a preferred embodiment, wherein the lentivirus is a Lenti-X HTX Packaging System (Clontech) was used to produce the lentivirus of Lenti-X Tet-On 3G, LV-TRE3G-NT-3-IRES-IL-10, and LV-TRE3G-LINGO-1-Fc-IRES-copGFP.

A preferred embodiment is directed towards a transgenic neural stem cell (NSC) engineered with a vector to insert cDNA corresponding to neurotrophin 3 (NT-3), IL-10, and LINGO-1, wherein said NSC to simultaneously produce a cocktail containing IL-10, neurotrophin3, and soluble LINGO-1 protein. In preferred embodiments, wherein said NSC comprises a lenti virus vector inserting the cDNA sequence corresponding to NT-3, IL-10, and LINGO-1. In preferred embodiments, wherein said NSC comprises SEQ ID Nos. 9, 10, and 11. In preferred embodiments, wherein SEQ IS Nos. 9, 10, and 11 are inserted using primers corresponding to SEQ ID Nos. 1-6. In preferred embodiments, wherein the NSC is suitable to inhibit pro-inflammatory M1 phenotype of microglia and switch it to M2 phenotype. In preferred embodiments, the transgenic NSC comprising a 90% conserved sequence with SEQ ID Nos 9, 10, and 11.

Use of any one of the therapeutics or methods for treatment of MS.

Use of the therapeutic of the above embodiments for expressing a therapeutic cocktail within a patient, wherein said therapeutic cocktail comprises NT-3, IL-10, and LINGO-1-Fc.

A further embodiment is directed towards a method of treating chronic stage EAE comprising administering to said patient a neural stem cell modified to express NT-3, IL-10 and LINGO-1-Fc.

A further embodiment is directed towards a method of treating multiple sclerosis comprising administering to said patient a transgenic neural stem cell expressing NT-3, IL-10, and LINGO-1-Fc. In preferred embodiments, the method comprising a transgenic neural stem cell sequences having 70% homology or greater to SEQ ID Nos. 9, 10, and 11. In preferred embodiments, the neural stem cell is modified as provided in the embodiments herein.

### BRIEF DESCRIPTION OF THE DRAWINGS

FIG. 1. NT-3/IL-10/LINGO-1-Fc promotes NSC proliferation and differentiation in vitro. (a) NSCs transduced with NT-3/IL-10/LINGO-1-Fc were plated on poly-D-lysine/laminin coated coverslips at a density of 2.0×104 cell/ml in 24 well plates and cultured in NSC proliferation medium. After 48 hours of culture with Dox (1.5 μg/ml), cells were stained with anti-Ki67 antibody. (b) Proliferation of NSCs was quantified as the percentage of Ki67-positive cells in all GFP-positive cells. (c) Examples of NSCs that differentiated into mature oligodendrocytes (MBP+), neurons (NF-H+), and astrocytes (GFAP+) in specific differentiation medium for 2 weeks. Scale bar=100 μm. (d) Quantitative analysis of differentiated cells. Data are shown as mean values±SD (n=5 each group) and are representative of three experiments. Groups designated by the same letter are not significantly different, while those with different letters (A, B, C, D, E or F) are significantly different (p<0.05-0.01), as determined by one-way ANOVA with Tukey's multiple comparisons test.

FIG. 2. Effect of cocktail-NSCs on the acute stage of EAE. (a) EAE mice were i.v. injected with various NSCs (1×106 cells/mouse) at onset of disease (day 10 p.i.) and then i.p. injected with Dox every two days (n=5 each group). Mice were scored blindly for disease severity daily by two researchers according to a 0-5 scale. (b) Accumulative score of EAE (sum of daily clinical scores from day 11 to day 28 p.i.). (c) Mice were sacrificed at day 28 p.i. (n=5 each group), and spinal cords were harvested for H&E stained spinal cord sections. The white matter of the lumbar spinal cord was analyzed to assess inflammation. (d) Mean score of inflammation in H&E staining. (e-g) Spinal cords and brains were harvested and MNCs isolated (n=5 each group). (e) Total MNC numbers in the CNS were counted under light microscopy. (f) Percentages of CD4+, CD8+, CD11b+, and CD11c+cells and (g) IFN-γ+, IL-17+, and GM-CSF+CD4+ T cells were determined by flow cytometry. Absolute numbers of different subtypes of CNS infiltrating cells were calculated by multiplying the percentages of these cells (as shown in FIG. 12) by total numbers of MNCs obtained from each spinal cord and brain tissue. Groups designated by the same letter are not significantly different, while those with different letters (A, B, C, D, E or F) are significantly different (p<0.05-0.01) as determined by one-way ANOVA with Tukey's multiple comparisons test. Data are presentative of 3 independent experiments.

FIG. 3. Effect of cocktail-NSCs on the chronic stage of EAE. (a) EAE mice were i.v. injected with NSCs (1×106 cells/mouse) at the chronic stage (60 p.i.) of disease and then i.p. injected with Dox every two days (n=5 each group). Mice were scored blindly for disease severity by two researchers according to a 0-5 scale. (b) Spinal cords were harvested from EAE mice described in FIG. 3a at day 120 p.i. for H&E staining (photos as shown in FIG. 14a), and inflammation was scored in white matter of the lumbar spinal cord and data were expressed as mean±SD. Spinal cords were immunostained against iNOS (c) and Arg1 (d) on infiltrating macrophages/microglia (Iba1+; photos as shown in FIG. 14b, c) and the numbers of double-positive cells were counted (e, g). Data represent mean±SD; n=5 mice per group. Groups designated by the same letter are not significantly different, while those with different letters (A, B, C, D, or E) are significantly different (p<0.05-0.01) as determined by one-way ANOVA comparison with Tukey's multiple comparisons test. Data are presentative of 3 independent experiments.

FIG. 4. Cocktail-NSCs reduced axon degeneration and astrogliosis at chronic stage of EAE. Mice described in FIG. 3a were sacrificed and lumbar spinal cords were harvested at day 120 p.i. (a) APP staining in the white matter lesion of lumbar spinal cord for detection of axon damage. (b) APP intensity in white matter lesions (%) was quantified using Image-Pro. APP intensity per field (%) was calculated using APP+ staining area divide the lesion area in the white matter. EAE lesions (lacking MBP labeling in FIG. 5c) were delimited by dashed lines. (c) GFAP staining of spinal cord sections for detection of astrogliosis. (d) Quantitative analysis of GFAP expression using Image-Pro. Data represent mean±SD; n=5 mice per group. Groups designated by the same letter are not significantly different, while those with different letters (A, B, C, D or E) are significantly different (p<0.05-0.01) as determined by one-way ANOVA comparison with Tukey's multiple comparisons test. Scale bar=100 μm. Data are presentative of 3 independent experiments.

FIG. 5. Cocktail-NSCs promote remyelination and MBP synthesis at chronic stage of EAE. Mice described in FIG. 3a were sacrificed and spinal cords were harvested at day 120 p.i. (a) LFB staining of spinal cord sections for detection of demyelination. (b) Image-Pro Plus software was used to manually outline the total white matter area (red trace). Blue area indicates intact myelin, whereas pale areas indicate demyelinated areas. Demyelination is expressed as percentage of the total demyelinated area out of the total white matter area. (c) MBP staining in the sham-EAE group compared with NSC-treated groups. (d) Quantitative analysis of MBP expression. MBP intensity was measured in the lesion areas in the lumbar spinal cord using Image-Pro. Data represent mean±SD; n=5 mice per group. Groups designated by the same letter are not significantly different, while those with different letters (A, B, C, D, E, F or G) are significantly different (p<0.05-0.01) as determined by one-way ANOVA comparison with Tukey's multiple comparisons test. Scale bar=100 μm. Data are presentative of 3 independent experiments.

FIG. 6. Toluidine blue staining for remyelination after NSC treatment at chronic stage of EAE. Mice described in FIG. 3a were sacrificed and spinal cords were harvested at day 120 p.i. (a) Toluidine blue staining of 1-μm semi-thin sections was performed to visualize myelination in naive and NSC-treated EAE mice. (b) All intact axons regardless of their myelination state, whose axoplasm was intercepted by a sampling line, were tagged and counted. (c) Mean g ratio (axon diameter divided by entire myelinated fiber diameter) was determined using ImageJ software as described (ref). Data represent mean±SD; n=5 mice per group. Groups designated by the same letter are not significantly different, while those with different letters (A, B, C, D or E) are significantly different (p<0.05-0.01) as determined by one-way ANOVA comparison with Tukey's multiple comparisons test. Data are presentative of 3 independent experiments.

FIG. 7. Quantitative analysis of neural cell differentiation of transplanted NSCs at chronic stage of EAE. Mice described in FIG. 3a were sacrificed at day 120 p.i. and the brains were harvested for histological staining. Striatum and corpus callosum were examined by immunohistology for neurons, OPCs, oligodendrocytes and astrocytes as shown in FIG. 14a-g. (a) Percentages of transplanted NSC (GFP+)—derived neurons (NeuN+), OPCs (A2B5+), mature oligodendrocytes (APC+), astrocytes (GFAP+) and undifferentiated NSCs (Sox2+) were shown. Data represent mean ±SD; n=5 mice per group. *p<0.05, **p<0.01. (b) Quantification of total APC+ and (c) NeuN+cell numbers. Data represent mean±SD; n=5 mice per group. Groups designated by the same letter are not significantly different, while those with different letters (A, B, C, D, E, F or G) are significantly different (p<0.05-0.01) as determined by one-way ANOVA comparison with Tukey's multiple comparisons test. Data are presentative of 3 independent experiments.

FIG. 8. Scheme of cocktail-producing NSCs in neural recovery in EAE. BBB: blood-brain barrier; Yellow: IL-10; Red: NT-3; Blue: LINGO-1-Fc. Arrow-headed or bar-headed line indicates promotion or inhibition, respectively.

FIG. 9. Generation of bone marrow derived NSCs in vitro. BM cells were isolated from adult C57BL/6 mice and cultured in DMEM/F-12 supplied with 20 ng/ml EGF, 20 ng/ml bFGF and 2% B27 supplements. Single cell were seeded in the poly-D-lysine and laminin coated plated. (a) After 1 week, the cells showed obvious proliferation ability. (b) After 2 weeks, the NSCs gradually formed neurospheres. (c) After 3 weeks, the neurosphere expanded with increasing the size and detached from the bottom of culture plate as free-floating spheres. (d,e) The neurospheres dissociated with accutase and plated in the coated coversplips were immunostained for NSC markers nestin and SOX2. Magnification×10 for d, ×20 for e. Scale bar=50 μm. Their proliferation and differentiation capacity was further determined in FIG. 1.

FIG. 10. Vector construction and NSC transduction. NSCs were prepared from bone marrow of C57BL/6 mice and, at the 5^{th}-10^{th} passages, transduced with 3 lentiviral vectors encoding tetracycline-controlled transactivator (TetOn), NT-3, IL-10, LINGO-1-Fc and copGFP. (a) Schematic structure of lentiviral vector. (I) Tet-On and neomycin, a selection marker conferring resistance to G418, were driven by EF1 promoter; (II), (III) The genes of NT-3/IL-10 or LINGO-1-Fc/copGFP expression were controlled by promoter TRE and the presence of Dox. These two vectors can confere resistance to puromycin and hygromycin. (b) Concentration of NT-3, IL-10 and LINGO-1-Fc in NSC supernatants of 48-hour culture with Dox (1.5 μg/ml) determined by ELISA. (c) Expression levels of Tet-On were detected by real-time PCR. Data represent mean±SD. b and c were repeated in 3 independent experiments. *p<0.001, statistical analyses was performed by one-way ANOVA comparison with Tukey's multiple comparisons test.

FIG. 11. Effect of cocktail-NSCs on CNS inflammation. (a) Schematic diagram of the measured area of spinal cord white matter. (b) Spinal cords were harvested from EAE mice described in FIG. 3a at day 120 p.i. for H&E staining. Scale bar=100 um. Spinal cords were also immunostained against iNOS (c) and Arg1 (d) on infiltrating macrophages/microglia (Iba1⁺). Scale bar=50 μm. Results were statistically analyzed as shown in FIG. 3b-d.

FIG. 12. Cocktail-NSCs are the most efficient in suppressing CNS inflammation. On day 28 p.i., MNCs were isolated from brain and spinal cords of cocktail-NSC-treated and control EAE mice. (a) Percentages of CD4⁺, CD8⁺ T cells, CD11b⁺ microglia/macrophage cells and CD11c⁺ dendritic cells were determined by flow cytometry. (b) Frequencies of IFN-γ⁺, IL-17⁺ and GM-CSF⁺ cells in gated CD4⁺ T cells were determined by flow cytometry. Statistical analyses are shown in FIGS. 2f-g. Data are presentative of 3 independent experiments.

FIG. 13. Cytokine production of MNCs from NSC-treated mice at acute EAE. Splenocytes harvested from mice with acute EAE, as described in FIG. 2a, were sacrificed at day 28 p.i. and stimulated with 25 μg/ml MOG35-55 for 3 days. Concentrations of IFN-γ, IL-17, GM-CSF, IL-5 and IL-10 in culture supernatants were measured by ELISA. Data represent mean±SD. Data are presentative of 3 independent experiments. Groups designated by the same letter are not significantly different, while those with different letters (A, B, C, D or E) are significantly different (p<0.05-0.01) as determined by one-way ANOVA comparison with Tukey's multiple comparisons test. Data are presentative of 3 independent experiments.

FIG. 14 Cocktail-NSCs reduce CNS inflammation and promote an M2 phenotype in macrophages/microglia. (a) Spinal cords were harvested from EAE mice described in FIG. 3a at day 120 p.i. for H&E staining. Scale bar=100 μm. Spinal cords were immunostained against iNOS (b) and Arg1 (c) on infiltrating macrophages/microglia (Iba1⁺). Scale bar=50 μm.

FIG. 15 Cocktail-NSCs inhibit microglia and astrocyte activation in vitro. Primary microglia (a) and astrocytes (b) were prepared from newborn B6 mice, stimulated with LPS (10 ng/ml and 1 μg/ml, respectively) and co-cultured with various NSC groups in transwells. Two days later, real-time PCR analysis of IL-10, iNOS, and TNF-a in microglia and GFAP, Vimentin, and N-cadherin in astrocytes was performed. Gapdh was used as an internal control. Data shown are mean±SD values obtained from three experiments. Groups designated by the same letter are not significantly different, while those with different letters (A, B, C, D or E) are significantly different (p<0.05-0.01) as determined by one-way ANOVA comparison with Tukey's multiple comparisons test.

FIG. 16. Neural cell differentiation of cocktail-NSCs in vivo. Mice described in FIG. 3a were sacrificed at day 120 p.i. and their brains were harvested. Striatum and corpus callosum were examined by immunohistology. (a) Representative colocalization of GFP⁺ cells with NeuN⁺ or Ga1C⁺ cells by confocal microscopy. (b-h) Immunofluorescence images. Co-localized GFP⁺ (green; transplanted NSCs) were identified with neural cell markers (red) of NeuN (b) and Tuj1 (c) for neurons, A2B5 (d) for OPCS, APC (e) and MBP (f) for oligodendrocytes, GFAP (g) for astrocytes and Sox2 (h) for undifferentiated NSCs. Shown are only control-NSC- and cocktail-NSC-treated mice as examples. Quantitative analysis is shown in FIG. 7 (n=5 mice per group). Data are presentative of 3 independent experiments.

FIG. 17. Cocktail-NSCs promoted primary OPC differentiation in vitro. OPCS were prepared from newborn B6 mice and cultured in oligodendrocyte differentiation medium for 48 h for O4 staining (a) and for one week for CNPase staining (d). Oligodendrocyte process was quantified as shown in (b). The ranges of process made by the extending processes with each circle of O4⁺ cells (c) and CNPase⁺ cells (e) were counted. (f) OPC proliferation (Ki67⁺) was reduced in differentiation medium plus cocktail-NSCs compared with differentiation medium alone. (g) Percentages of Ki67⁺ cells. Data represent mean±SD; Data were performed in 3 independent experiments. Groups designated by the same letter are not significantly different, while those with different letters (A, B, C, D or E) are significantly different (p<0.05-0.01) as determined by one-way ANOVA comparison with Tukey's multiple comparisons test. Scale bar=100

### DETAILED DESCRIPTION OF THE EMBODIMENTS

In the present study, we have tested the effect of a novel system on the chronic stage of EAE using adult NSCs that were engineered to simultaneously produce a therapeutic cocktail (cocktail-NSCs) containing IL-10, an effective immunoregulatory cytokine; neurotrophin 3 (NT-3), a potent neurotrophic factor; and soluble LINGO-1 protein (LINGO-1-Fc), an antagonist of LINGO-1, a key part of the common receptor complex for neuroregeneration inhibitors [24], under the control of the Tet-on system. These NSCs target the three major mechanisms underlying the pathogenesis of chronic/progressive EAE and would thus be a highly effective approach for EAE therapy. This cocktail, continuously produced by autologous NSCs on the CNS disease foci, would also avoid potential side effects caused by repeated systemic administration of a large amount of drugs, or harmful local administration [25].

### Generation and Characterization of Bone Marrow (BM) Derived NSCs

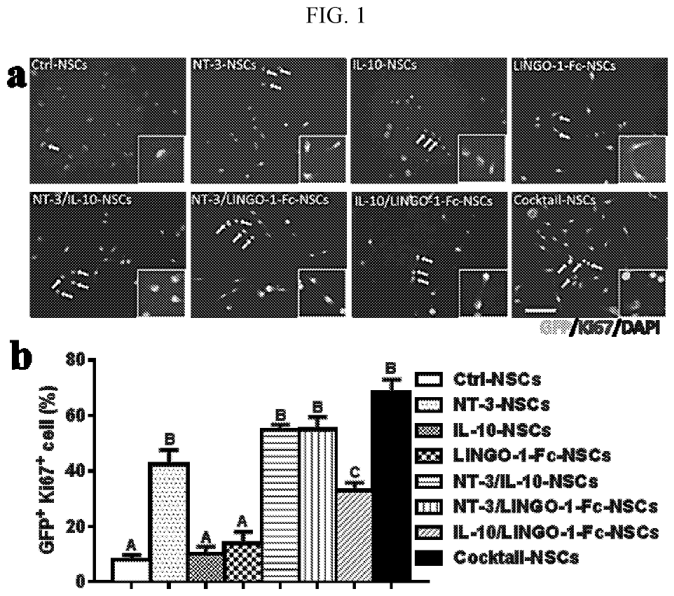

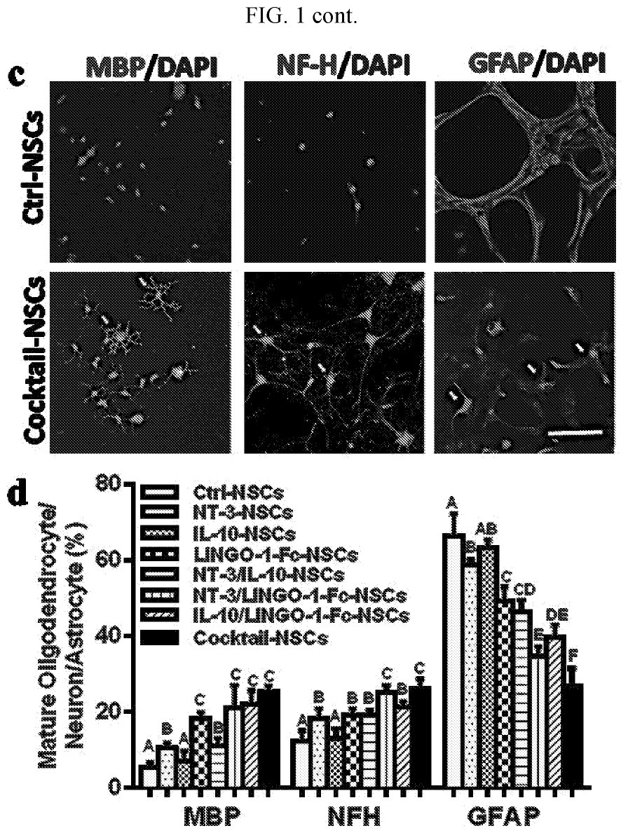

BM cells were isolated from femurs of the adult B6 mice as described in Materials and Methods and our previous report [22]. After 1 week in culture, individual cells exhibited obviously proliferative ability as shown in FIG. 9a. Two weeks later, individual cells proliferated to form various sizes of neurospheres (FIG. 9b) and, at 3-4 weeks, the neurosphere expanded with increasing the size and gradually detached from the bottom of culture plate as free-floating spheres (FIG. 9c). These neurospheres were collected, dissociated to single cells, and re-seeded at 1.0×105 cells/ml for next round of or expansion. Neurospheres at 5th passage were dissociated into single cells, transferred into poly-D-lysine and laminin pre-coated coverslips and used in all in vitro experiments. These cells were positive for NSC markers nestin and SOX2 (FIG. 9d, e), and the NSC property was further confirmed by their capacity to proliferate and differentiate into neural cell lineages (FIG. 1).

### Transduction of NSCs with Inducible NT-3, IL-10 and/or LINGO-1-Fc

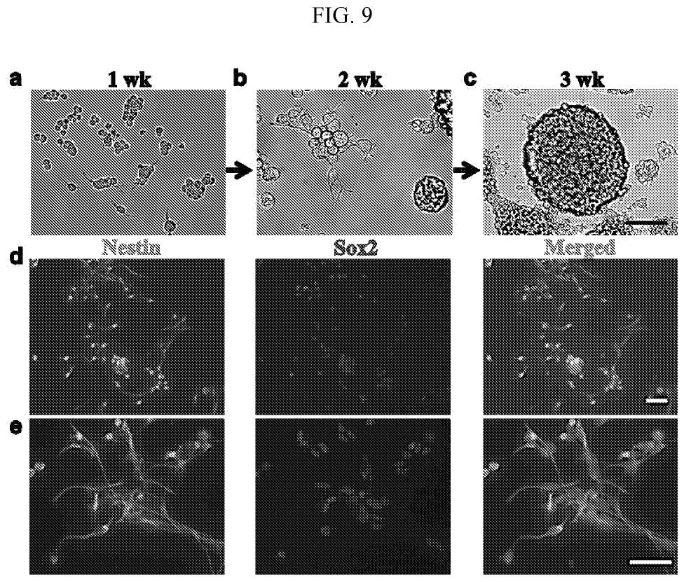

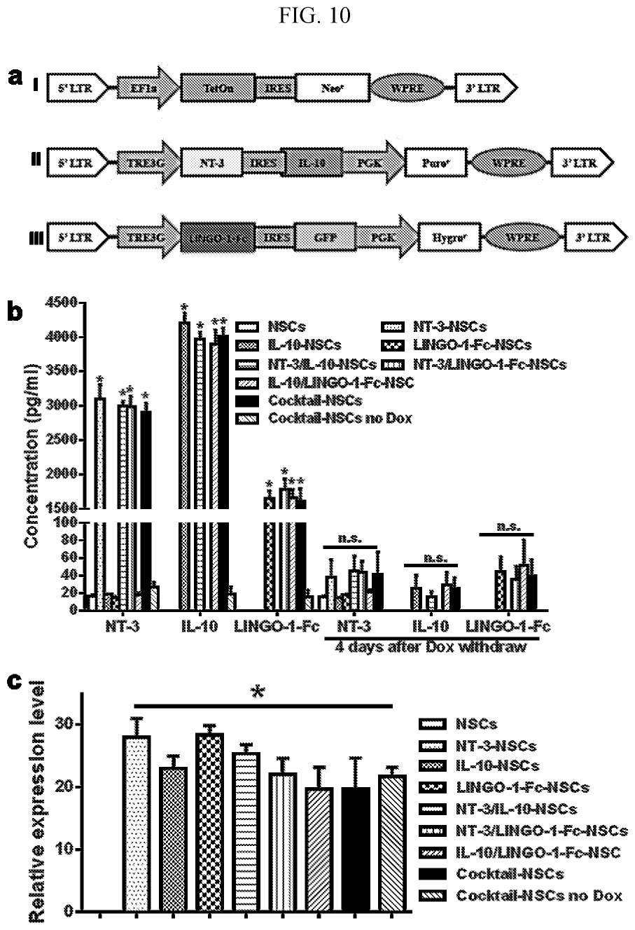

The most recent Lenti-X™ Tet-On® Advanced Inducible Expression System was used to transduce tetracycline-controlled trans activator, NT-3, IL-10, LINGO-1-Fc and copGFP into NSCs cells. Innovatively, we inserted NT-3, IL-10, and LINGO-1-Fc genes individually into separate vectors and transduced them into cells sequentially. This method avoids the drawback of limited space for insertion of several genes in a lentiviral vector. We used neomycin, puromycin and hygromycin as selection markers in different vectors to ensure that genes are transduced into the same cell. The structure of the vectors is shown in FIG. 10a.

To test the expression of these transduced genes in NSCs, we cultured the cells with or without Dox [26]. After 24 hours, supernatants and cells were harvested and analyzed by ELISA and real-time PCR. NT-3, IL-10, and LINGO-1-Fc were strongly expressed only in the presence of Dox (FIG. 10b). Meanwhile, cells transfected with a single gene (NT-3-NSCs, IL-10-NSCs, and LINGO-1-Fc-NSCs) or two genes (NT-3/IL-10-NSCs, NT-3/LINGO-1-Fc-NSCs, and IL-10/LINGO-1-Fc-NSCs) also produced significant levels of corresponding proteins. Withdrawal of Dox led to a decrease in the production of these proteins after 4 days (FIG. 10b), confirming the efficacy of the Tet-on/off system. Furthermore, Tet-On 3G mRNA was present, as detected by real-time PCR, in NSCs transduced with these lentiviruses (FIG. 10c). These results confirm the success of gene transduction and the Tet-on system.

### Proliferation and Differentiation Potentials of Cocktail-NSCs In Vitro

The proliferation capacity of cocktail-NSCs was tested by Ki67 immunostaining. Interestingly, the number of Ki67+ cells was significantly increased in all NSCs that carried the NT-3 gene (NT-3-NSCs, NT-3/IL-10-NSCs, NT-3/LINGO-1-Fc-NSCs, and cocktail-NSCs) (FIG. 1a, b). Adding Dox to mock-transduced control NSCs did not change their proliferation.

To investigate their effect on NSC differentiation, NT-3, IL-10 and/or LINGO-1-Fc-transduced NSCs were cultured in specific differentiation medium with or without Dox treatment. After 2-3 weeks, a number of NSCs had differentiated into astrocytes, neurons, or oligodendrocytes, while adding Dox to the control-NSCs did not change their differentiation profile. NSCs transduced with NT-3 and LINGO-1-Fc differentiated into higher numbers of neurons and oligodendrocytes, but fewer astrocytes (FIG. 1c, d). These results indicate that NT-3 and LINGO-1-Fc promote differentiation of oligodendrocytes and neurons, while reducing differentiation of astrocytes.

### Cocktail-NSCs Suppress Acute EAE

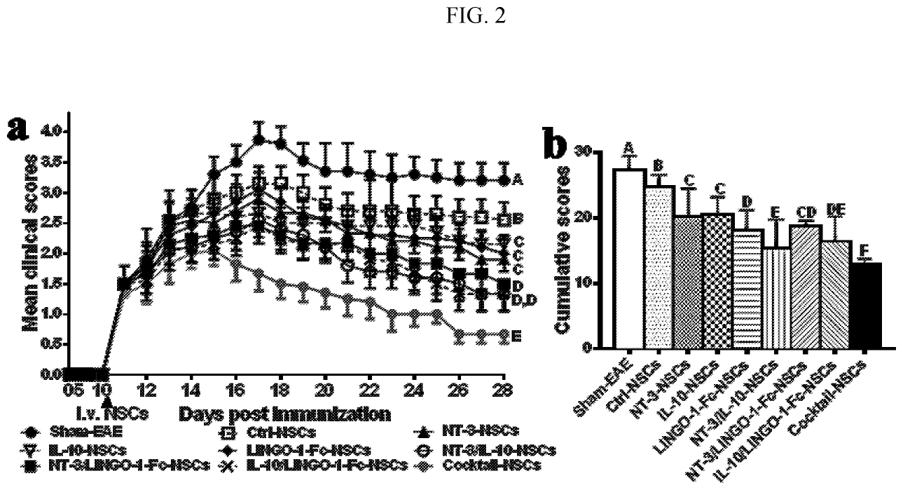

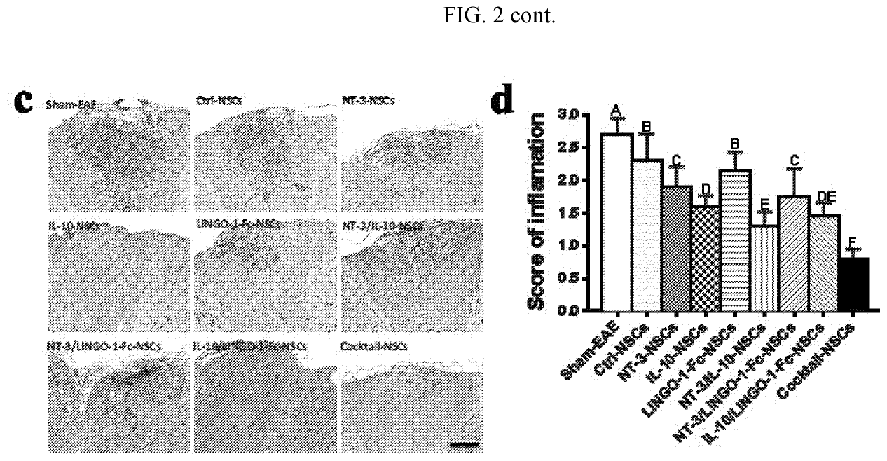

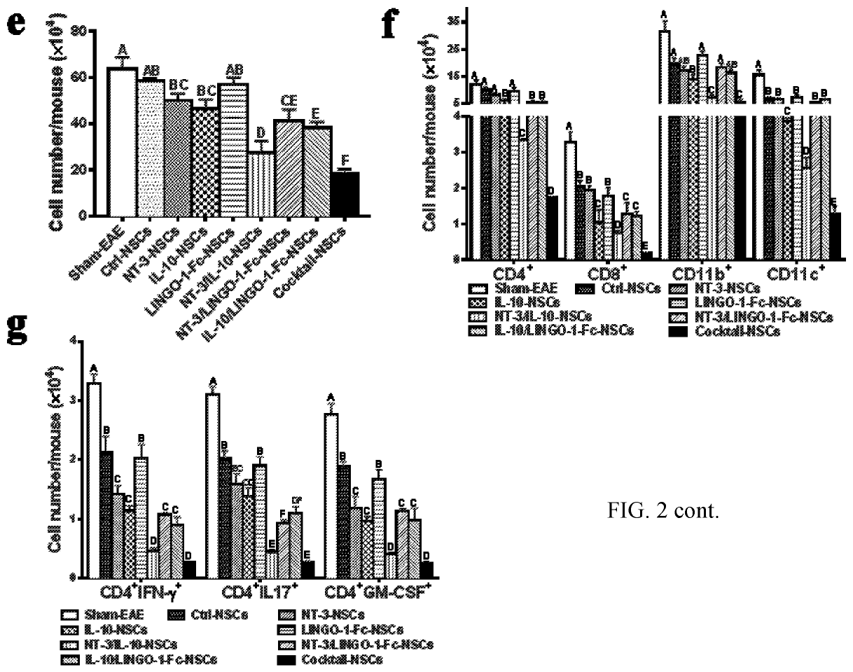

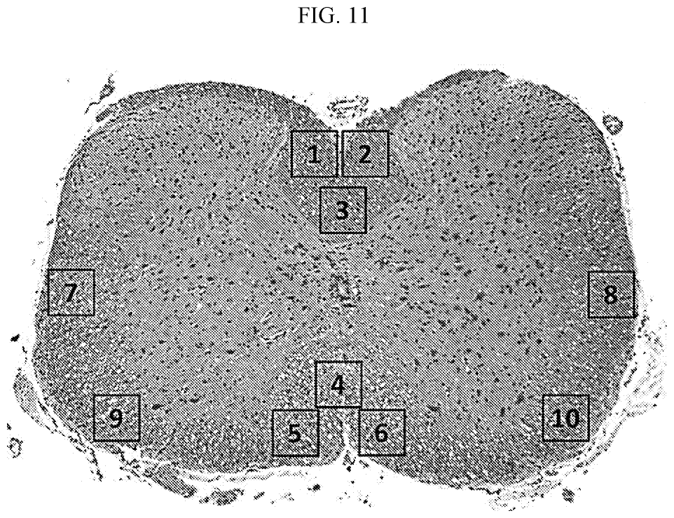

In order to evaluate the effect of cocktail-NSCs on the development of EAE, we first i.v. injected cocktail-NSCs (1.0×106 cell/mouse) into mice at disease onset (day 10 p.i.). The group treated with PBS (Sham-EAE) showed a progressive increase in clinical score from days 10 to 17 p.i., while mice treated with control NSCs, as well as those treated with NSCs transduced with one or two genes, showed a significant reduction in clinical scores at day 28 p.i. compared with the Sham-EAE group (p<0.05-0.01). Cocktail-NSC-treated mice exhibited the best recovery (p<0.001), indicating that therapeutic effect of cocktail-NSCs was significantly more potent than that of NSCs transduced with a single gene (p<0.01) or with two genes (p<0.05) (FIG. 2a, b). Consistent with the clinical score, sham-EAE mice showed severe EAE histological alterations, while only few inflammatory infiltrates were present in the spinal cord white matter of the cocktail-NSC-treated mice (p<0.01; FIG. 2c, d), as measured in different white matter areas (FIG. 11). Meanwhile, mice treated with NSCs transduced with a single gene or two genes also exhibited different degrees of recovery, and the treatment groups with NSCs producing IL-10 recovered better than those without IL-10 (p<0.05-0.01; FIG. 2c, d). These results indicate that overexpression of IL-10 is necessary for the anti-inflammatory effects of cocktail-NSCs.

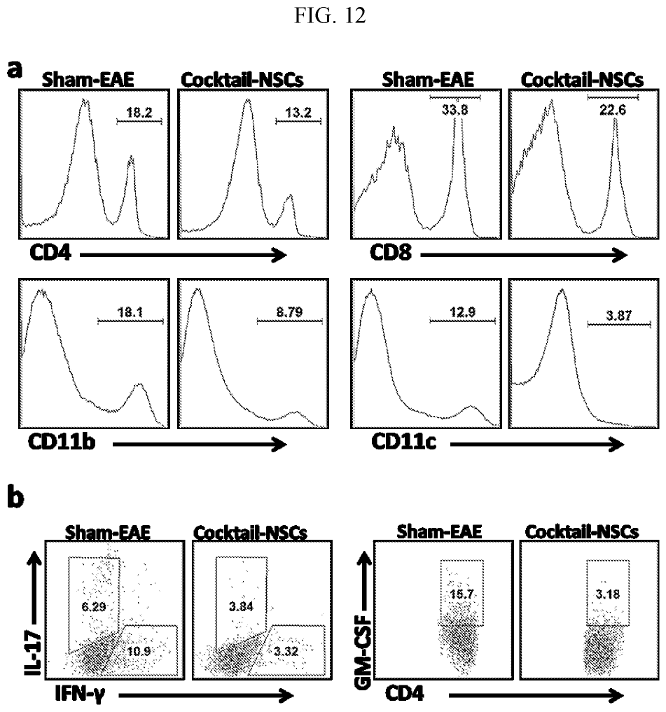

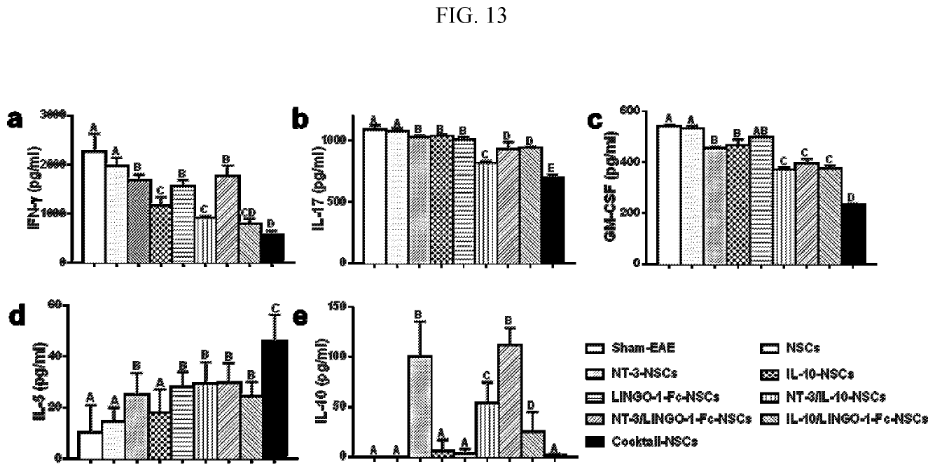

To assess the therapeutic effect of cocktail-NSCs on the infiltration of pathogenic T cells into the CNS, MNCs were isolated from the CNS and analyzed by flow cytometry. The total number of MNCs was 6.3×105 per mouse in the sham-EAE group vs. 1.9×105 in the cocktail-NSC-treated group. The NT-3/IL-10-NSC-treated group also showed fewer infiltrating cells (2.9×105 cell/mouse) compared with the sham-EAE group (FIG. 2e). Significantly fewer CD4+ T cells and CD11c+ dendritic cells were observed in the CNS of cocktail-NSC-treated EAE mice than in other groups (FIG. 2f, FIG. 12a). Further, cocktail-NSC treatment significantly reduced the percentages of Th1 (CD4+IFN-γ+) and Th17 (CD4+IL-17+) cells in the CNS compared with control (p<0.01-0.001; FIG. 2g, FIG. 12b). GM-CSF, a critical cytokine in Th17 cell pathogenicity [27], was also significantly decreased by cocktail-NSC treatment (p<0.001). Furthermore, the percentages and absolute numbers of IFN-γ+CD4+, IL17+CD4+ and GM-CSF+CD4+ cells were also significantly decreased in all groups treated with NSCs producing IL-10. These results indicate that IL-10 in the cocktail plays an important role in the inhibition of CNS inflammatory infiltration.

We and others have shown that NSCs, upon i.v. injection in EAE mice, were found in almost all peripheral organs within 10 days, and subsequently completely disappeared from them by day 30 after transplantation (Pluchino Nature 2003, 2005, Yang JCI, 2009, Einstein O, Ann Neurol. 2007 March; 61(3):209-18.), and migrate exclusively into the CNS inflamed foci (Ben-Hur T, Glia. 2003; 41(1):73-80). During their stay in the periphery, these cells played an immunomodulatory role, primarily by inducing Th1, but not Th2, cell apoptosis (Pluchino Nature 2005), and transduction with IL-10 significantly enhanced this effect (Yang, J C I, 2009).

To study the autoantigen-induced cytokine production of cocktail-NSC-treated mice, splenocytes were harvested 28 days p.i. and stimulated with MOG35-55. As shown in FIG. 13, concentrations of IFN-γ in cell culture supernatants were dramatically decreased in all groups treated with NSCs overexpressing IL-10. Injection with NT-3/IL-10-NSCs and cocktail-NSCs significantly decreased IL-17 production, and the GM-CSF level was also obviously decreased in mice treated with cocktail-NSCs. Treatment with NT-3-NSCs and NT-3/IL-10-NSCs showed a much higher IL-10 level than in other groups. There was no significant difference in proportions of various immune cell proportions among splenocytes of these groups (data not shown). Overall, cocktail-NSCs inhibited IFN-y, IL-17 and GM-CSF production.

### Cocktail-NSCs Significantly Alleviate Chronic Stage of EAE

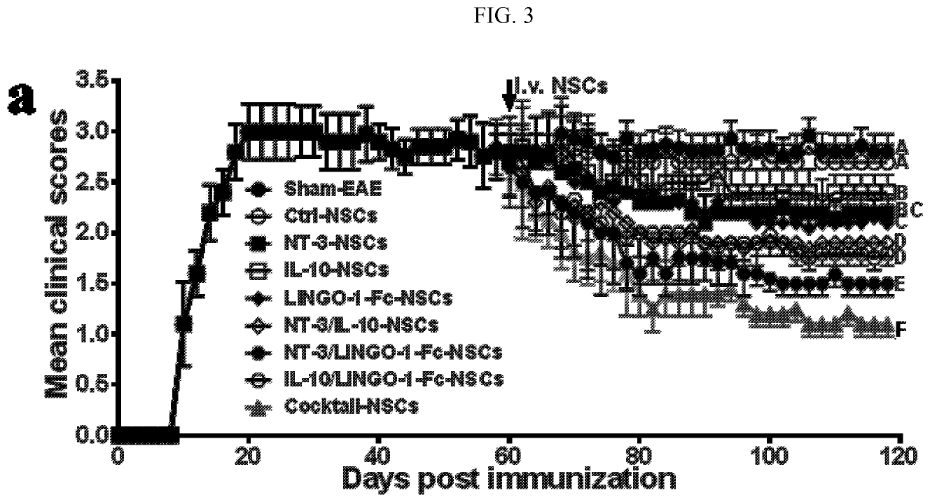

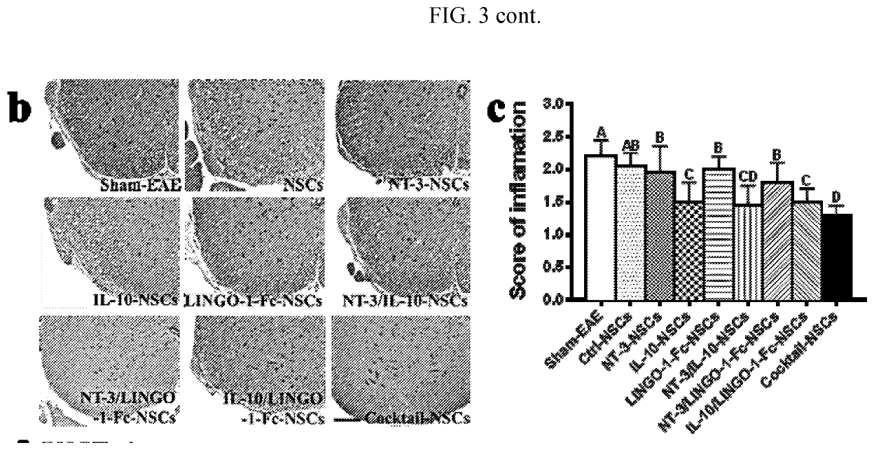

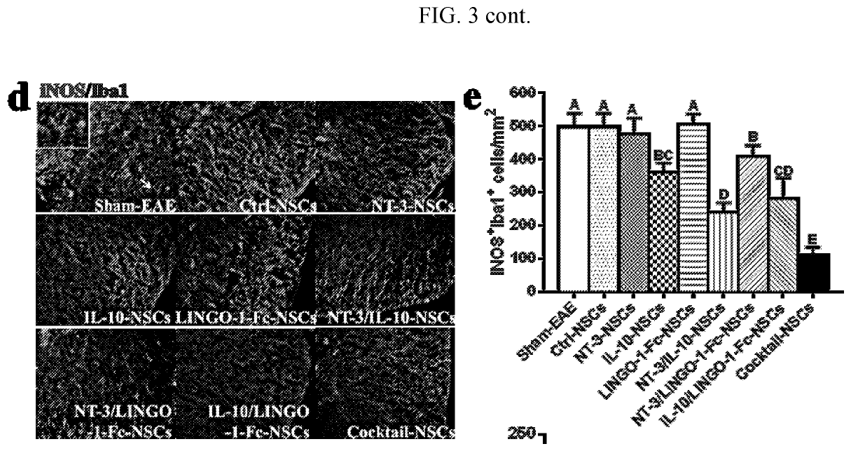

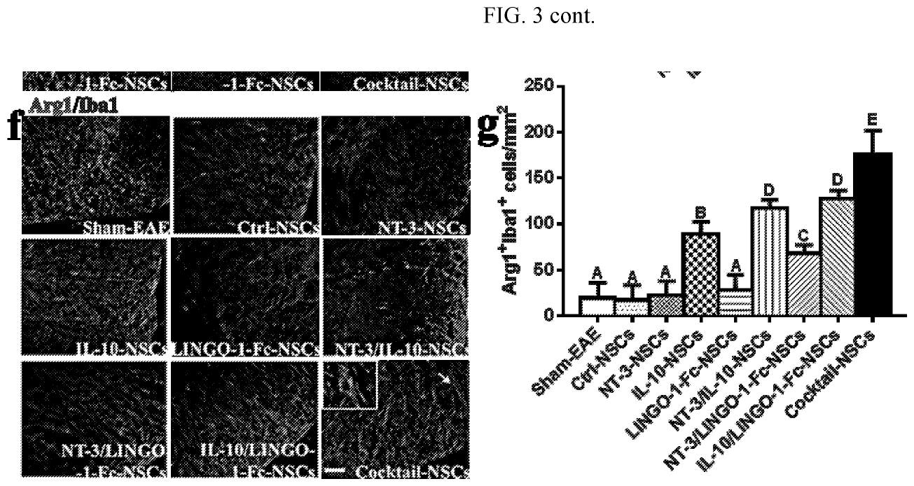

To define the effect of therapeutic cocktail-NSCs on the chronic stage of EAE, mice were transplanted with cocktail-NSCs by i.v. injection at day 60 p.i. PBS-injected mice exhibited a slight recovery at acute phase, and then remained stable up to day 120 p.i. (FIG. 3a), similar to described previously [28]. While control NSCs showed no effect when treatment started at the chronic stage (FIG. 3a), NSCs transduced with a single gene (NT-3- or LINGO-1-Fc-NSCs alone, but not IL-10 alone) slightly halted disease progression compared with the sham-EAE group at day 120 p.i. (p<0.05). The mean clinical score demonstrated that cocktail-NSCs and NT-3/LINGO-1-Fc-NSCs significantly suppressed EAE development at chronic stage EAE. In addition, the cocktail-NSC-treated group also showed significantly inhibited EAE compared with mice treated with NT-3/IL-10-NSCs or IL-10/LINGO-1-Fc-NSCs. These results indicate that, compared to un-modified NSCs or cells transduced with one or two therapeutic molecules, the cocktail-NSCs were the most effective at alleviating EAE when treatment was started at chronic stage, e.g., day 60 p.i.

Given that the autologous property of BM-NSCs makes them of great interest for future clinical use, we focused on these cells in the current study. Similar results have also been observed in NSCs derived from the subventricular zone (SVZ) of brain (data not shown).

### Cocktail-NSCs Reduce CNS Inflammation and Promote an M2 Phenotype in Macrophages/Microglia

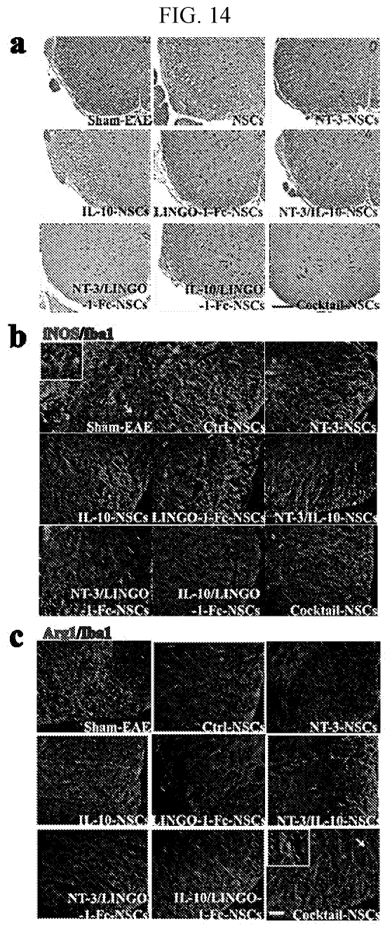

In order to determine the anti-inflammatory capacity of cocktail-NSCs at the chronic stage of EAE, H&E staining was performed to detect the extent of CNS inflammation. While IL-10-producing NSCs had stronger anti-inflammatory effects than those without IL-10, cocktail-NSC-treated mice showed the lowest inflammation score compared with all other groups (FIG. 14a; FIG. 3b; p<0.05-0.01).

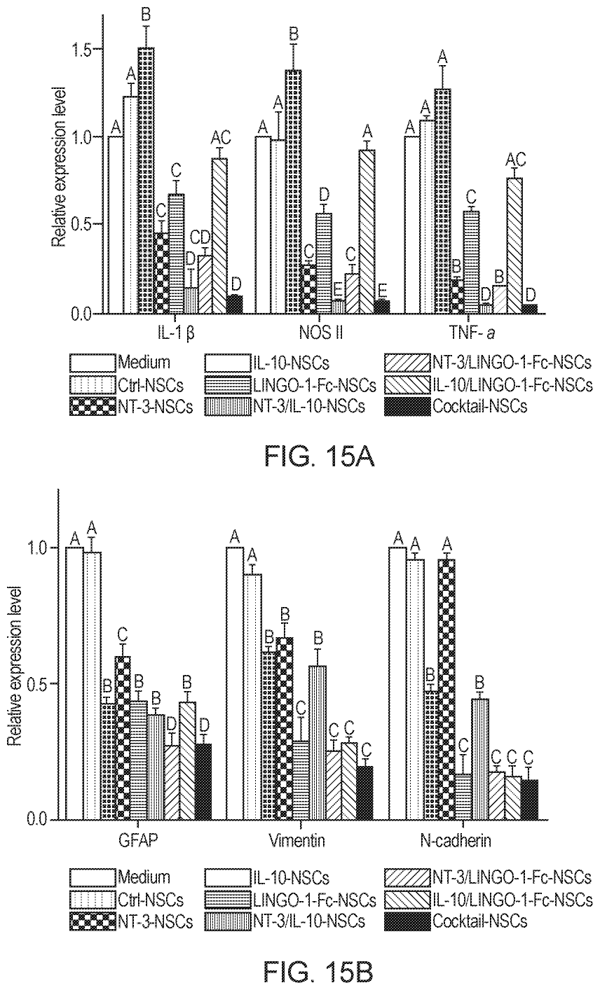

Microglia/macrophages can exhibit either pro- or anti-inflammatory properties, depending on the disease stage and the signals they receive [29]. Here, we determined expression of iNOS and Arginase-1 (Arg1), primary markers of Type 1 (M1; pro-inflammatory) and Type 2 (M2; immunomodulatory), respectively, by macrophages/microglia (Iba1+) in the spinal cord. Higher numbers of iNOS+Iba1+(FIG. 14b; FIG. 3c) and lower of Arg1+Iba1+ cells (FIG. 14c; FIG. 3d) were observed in the sham-EAE group compared to other groups, while cocktail-NSC-treated mice had more Arg1+ and fewer iNOS+Iba1+ cells. We also tested whether these engineered NSCs modulate microglial activation induced by lipopolysaccharide (LPS) treatment in vitro. In primary microglial cultures, cocktail-NSCs, as well as other IL-10-producing NSC groups, effectively inhibited expression of major mediators of microglia activation, e.g., TNF-α, IL-1β, and iNOS (FIG. 15a). These results indicate that cocktail-NSCs can inhibit the pro-inflammatory M1 phenotype of microglia and switch it to anti-inflammatory or immunoregulatory M2 phenotype, which can stimulate the remyelination process [30].

### Cocktail-NSCs Reduce Axon Degeneration and Astrogliosis

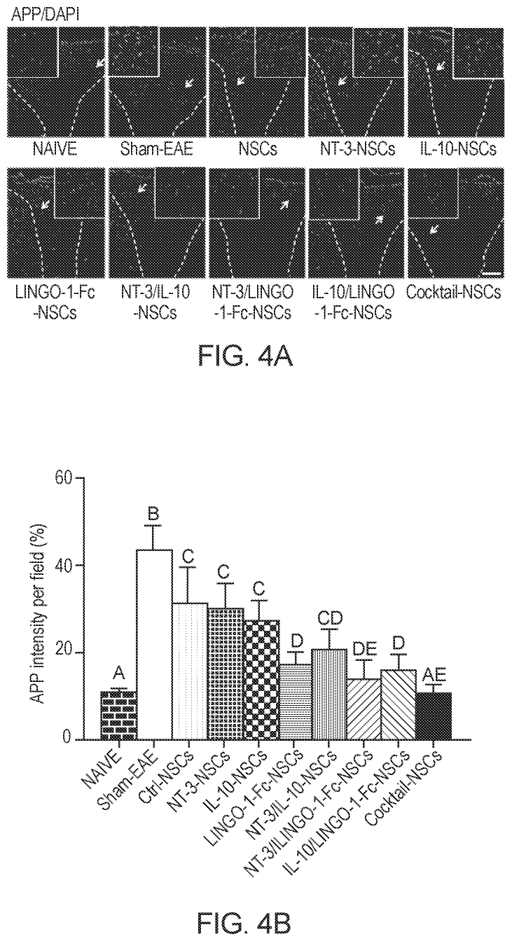

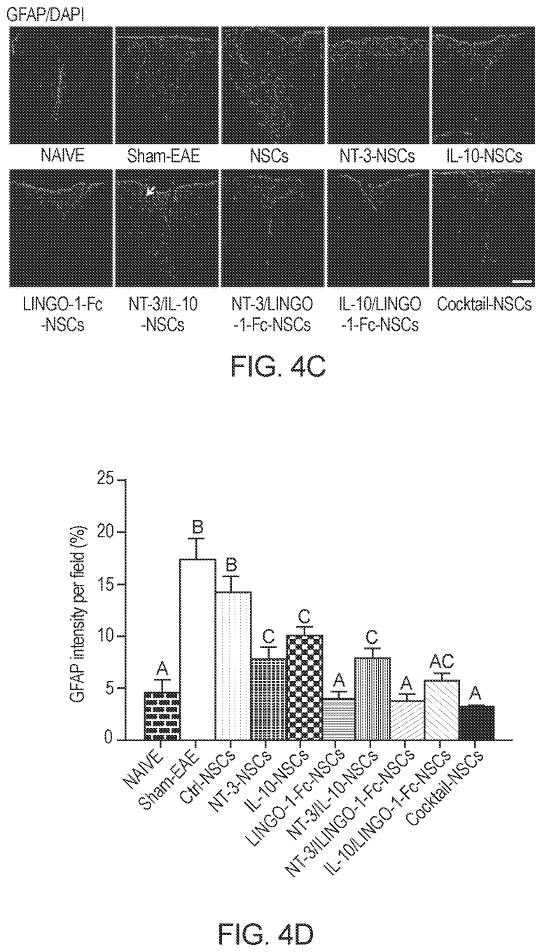

It is believed that the damage of myelin sheaths in EAE is greatly harmful for axonal survival [31], vice versa, and axonal injury precludes remyelination due to the alteration of remyelination signals from axons [32]. Therefore, treatment that could preserve axonal integrity of EAE mice may be of potential as a neuroprotective agent. Here, axonal pathology of mice treated at the chronic stage of EAE was evaluated by the development of amyloid precursor protein (APP) deposits, a prototypical marker of axonal damage [33]. In comparison with naïve mice, sham-EAE mice exhibited high intensity of APP stainings in the white matter of spinal cord (FIG. 4a). Mice that received NSCs transduced with IL-10 or NT-3 alone showed a small but significant decrease in APP stainings when compared with sham-EAE mice. All groups treated with NSCs transduced with LINGO-1-Fc had lower APP+stainings than those without LINGO-1-Fc (p<0.05; FIG. 4b). The lowest intensity of APP stainings was observed in cocktail-NSC-treated EAE mice, indicating the potential effects of cocktail-NSCs in axonal protection, in which LINGO-1-Fc played a major role.

Dense astrogliosis is an important factor in plaque formation and MS chronicity [34]. We thus examined the effects of cocktail-NSCs on astrogliosis in EAE spinal cord sections using immunolabeling for GFAP. There was a more than fivefold reduction in reactive astrogliosis in mice that received cocktail-NSCs compared to sham-EAE. Astrogliosis was also significantly attenuated in mice treated with LINGO-1-Fc-NSCs (FIG. 4c, d). These findings are in agreement with our in vitro data, which showed that LINGO-1-Fc dramatically suppressed expression of astrocyte genes, GFAP, Vimentin, and N-cadherin, in response to LPS activation (FIG. 15b). Together, these results indicate that cocktail-NSCs effectively reduce axonal injury and astrogliosis.

### Cocktail-NSCs Promote Remyelination

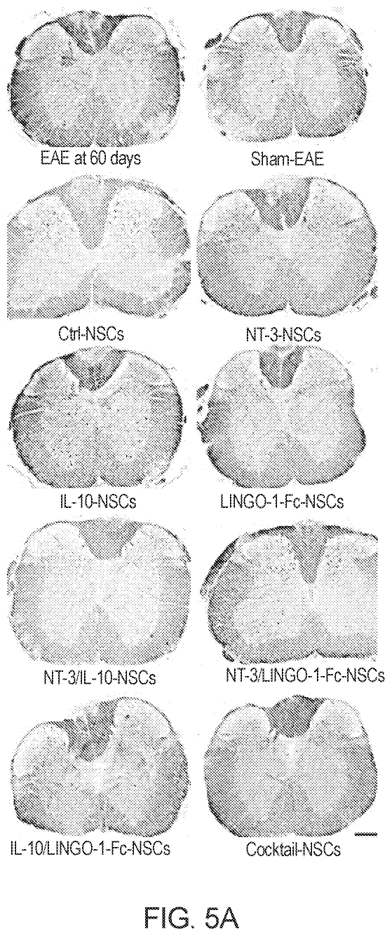

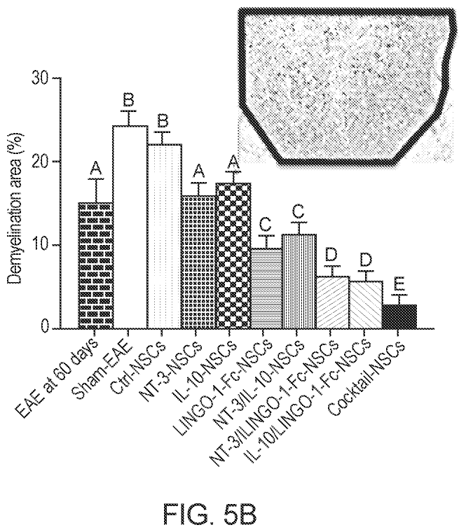

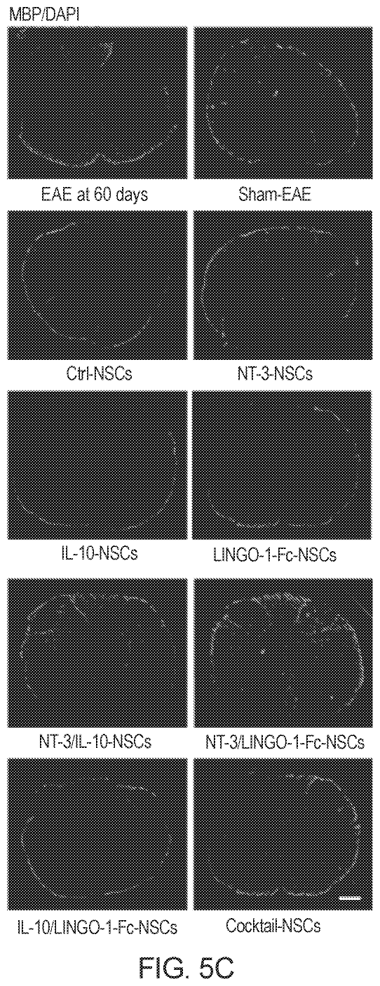

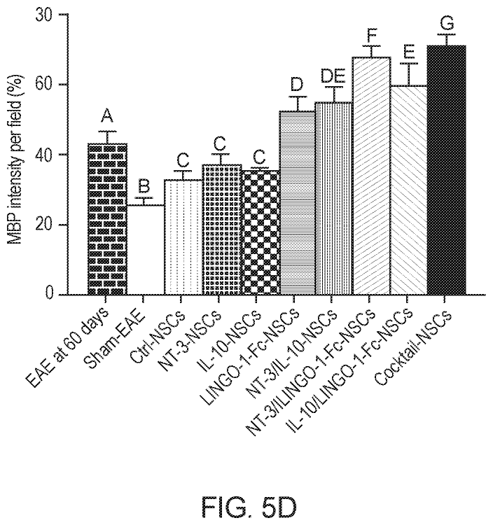

To characterize possible remyelination, EAE mice were treated with different groups of NSCs starting at day 60 p.i., when chronic damage in the CNS was established. Lumbar spinal cords of a group of mice were harvested this day (before treatment) for Luxol fast blue (LFB) and MBP stainings, which served as a baseline of demyelination. As shown in FIG. 5a-d, a significant degree of demyelination had occurred by day 60 p.i. (before treatment; baseline), and the degree worsened in sham-EAE mice up to day 120 p.i., indicating a progressive demyelination. EAE mice received NSCs expressing either NT-3 or IL-10 alone showed a similar demyelination level compared to the baseline, indicating blockade of further demyelination but no remyelination. Importantly, NSCs transduced with LINGO-1-Fc, or any two therapeutic genes, significantly reduced demyelination and enhanced MBP expression compared to the baseline (before treatment), and cocktail-NSCs had the strongest effects, providing evidence for not only blockade of further demyelination but also successful remyelination. Taken together, our data demonstrated that the enhanced myelination observed in mice receiving cocktail-producing NSCs is due to both the blockade of further demyelination and the promotion of remyelination.

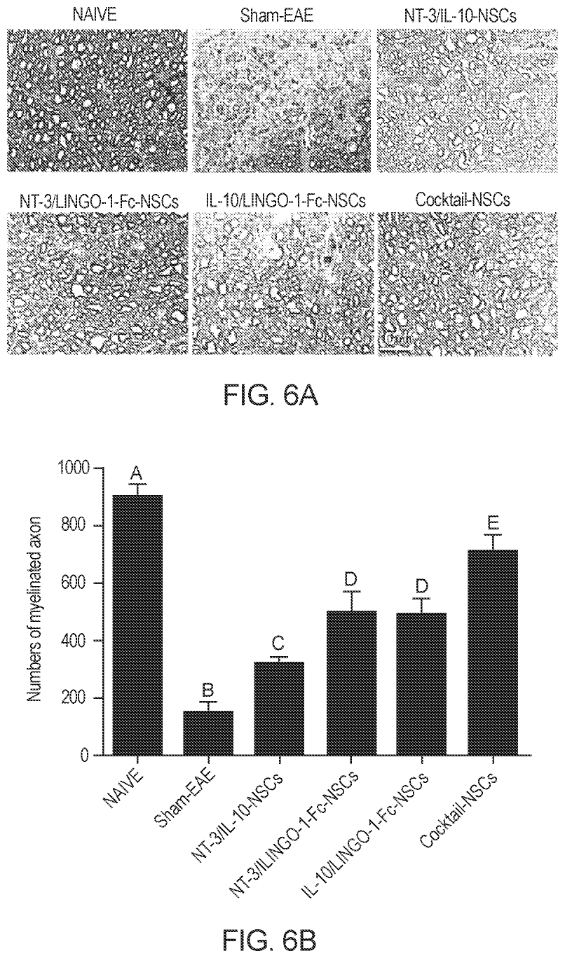

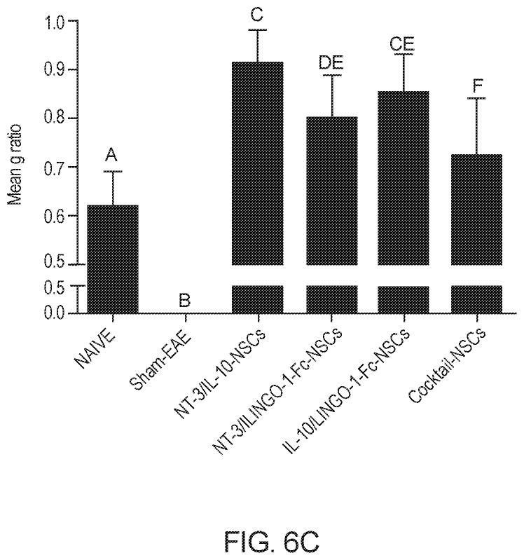

Toluidine blue-stained sections were also used to visualize myelinated axons (FIG. 6a). Loss of myelin was apparent in spinal cords of sham-treated mice, whereas a greater number of myelinated and/or remyelinated axons was found in cocktail-NSC-treated mice. Newly formed myelin sheaths were thinner than in naive mice (FIG. 6b). Consistent with the neuropathy phenotype, the g-ratio (measuring the size ratio between diameter of axon and total fibre) of the cocktail-NSC-treated group was significantly lower than the sham-EAE and other NSC-treated groups (FIG. 6c), a feature of enhanced remyelination [6, 8]. These results clearly indicate that treatment with NSCs that produce therapeutic cocktail not only prevents further CNS tissue damage but, importantly, also induces remyelination and neurorepair in chronic EAE.

### Cocktail-NSC Treatment Increases the Numbers of Neurons and Oligodendrocytes

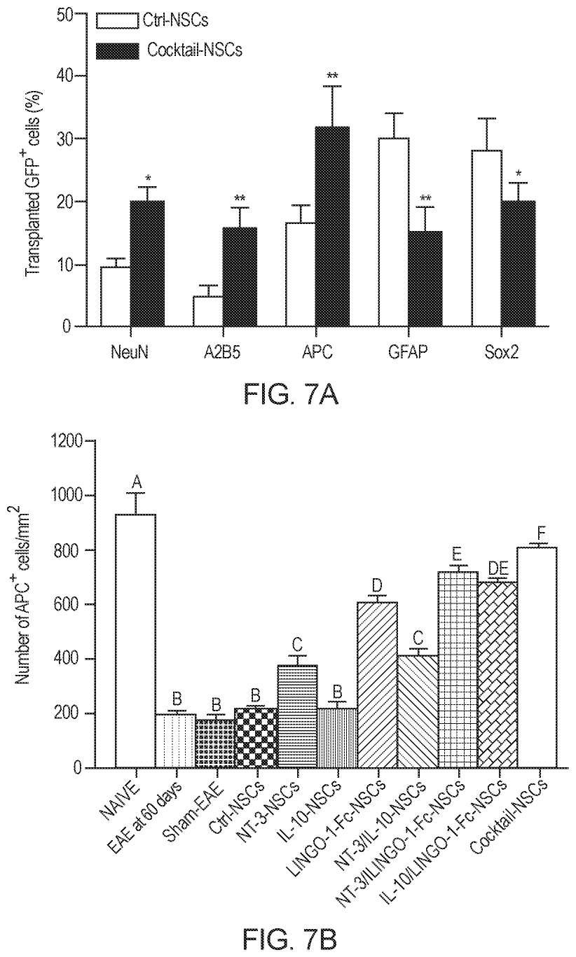

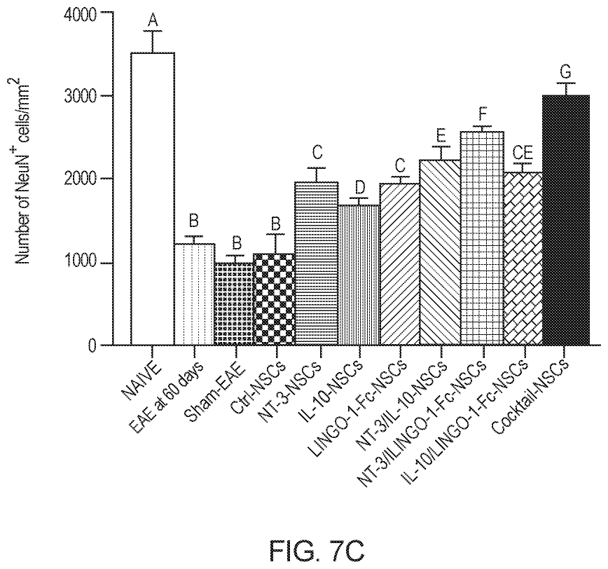

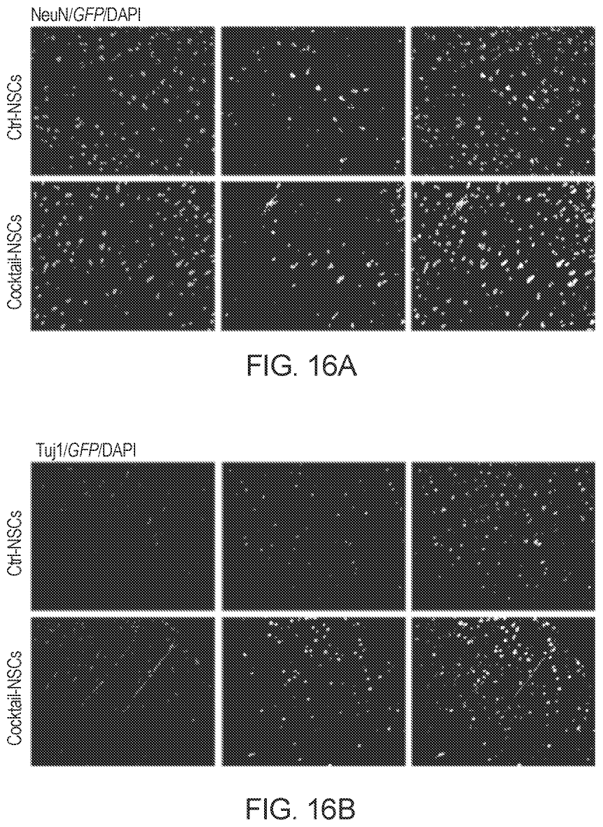

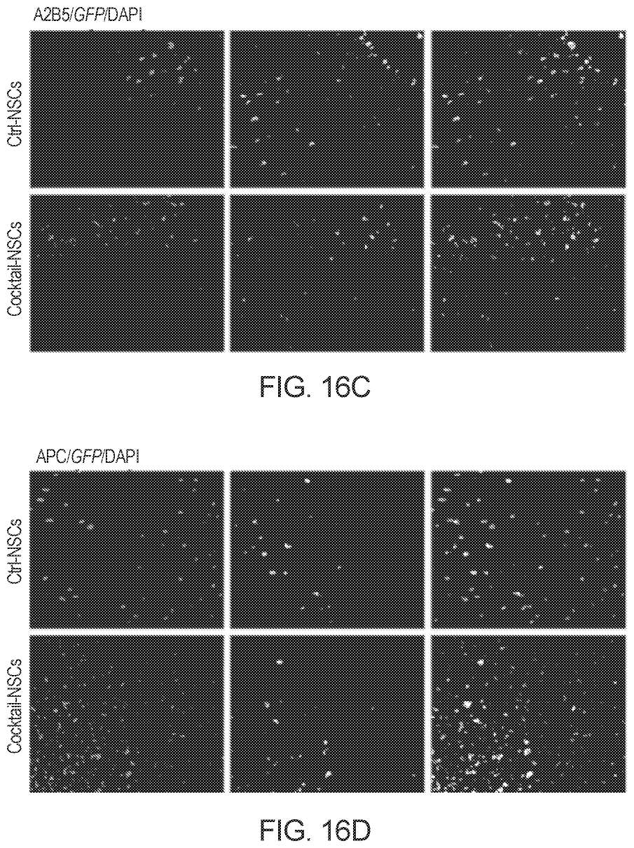

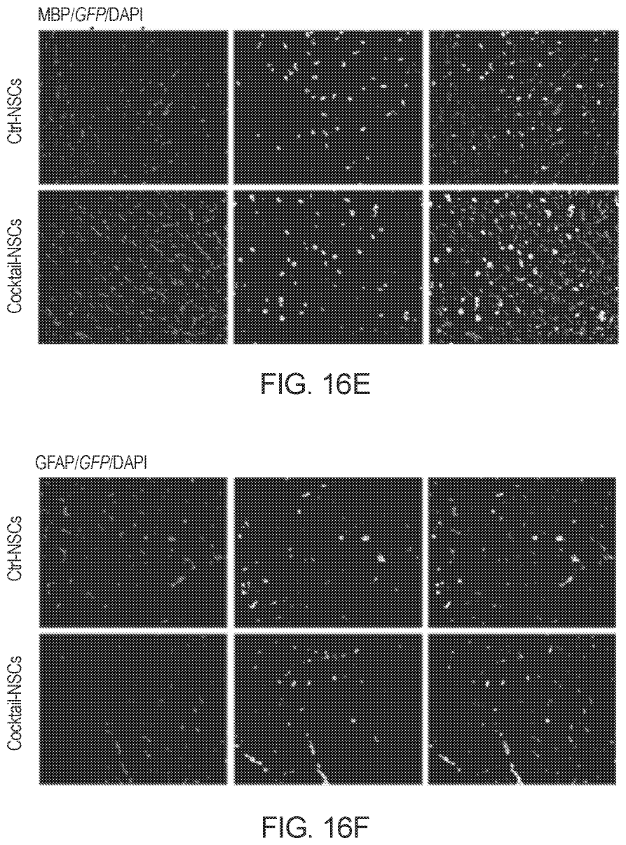

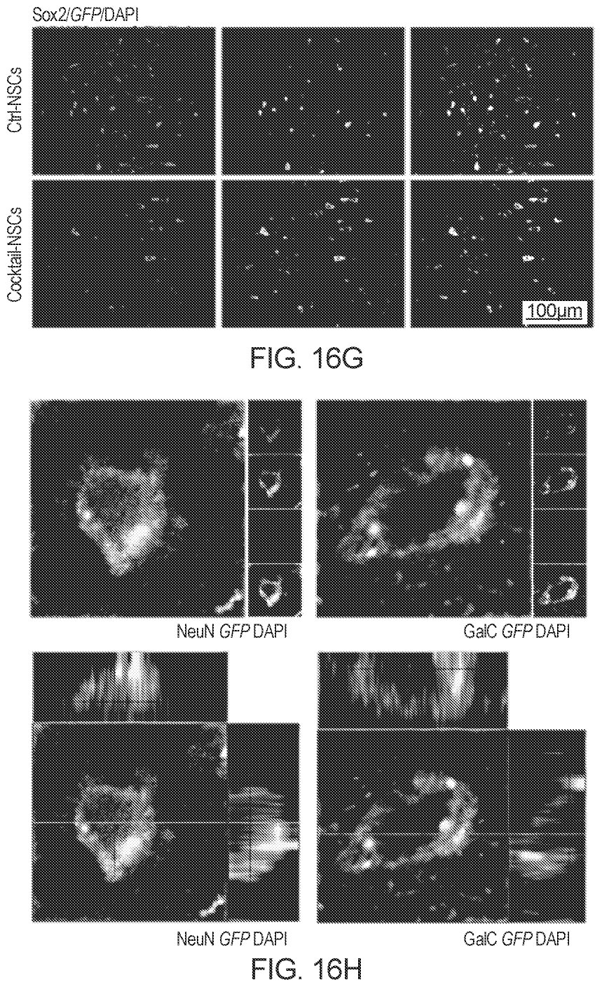

We then studied the effect of the cocktail on neural cell differentiation of transplanted NSCs in demyelinated lesions by triple immunostaining. Co-localization of GFP (for transplanted NSCs) and neural specific markers in the striatum and corpus callosum (as shown as FIG. 16a) revealed that some of the transplanted cells differentiated into NeuN+ and Tuj1+ neurons (FIG. 16b, c), A2B5+ OPCs (FIG. 16d), APC+ and MBP+ mature oligodendrocytes (FIG. 16e, f), GFAP+astrocytes (FIG. 15g), while some NSCs remained undifferentiated (Sox2+; FIG. 16h). Quantitative analysis showed that significantly higher percentages of neurons, OPCs and oligodendrocytes and a smaller percentage of GFAP+ astrocytes were derived from transplanted cocktail-NSCs than from control-NSCs (p<0.05-0.01) (FIG. 7a-c). While up to 30.3±1.1% of transplanted control NSCs (GFP+) retained undifferentiated Sox2+ morphological features (FIG. 16h), consistent with a previous report [16], a significantly smaller percentage (20.5±2.0%) of these cells was observed in cocktail-NSCs (FIG. 7a), indicating that a large proportion of cocktail-NSCs had differentiated into neural cells. Importantly, the majority of the MBP-stained area and neural cells were GFP-, indicating that the main mechanism of cocktail-induced remyelination and neural repair is via promotion of endogenous repair.

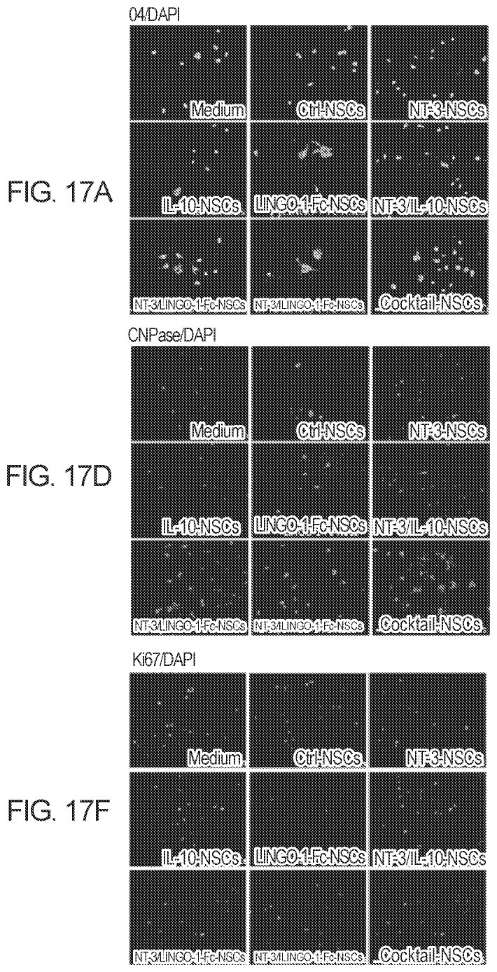

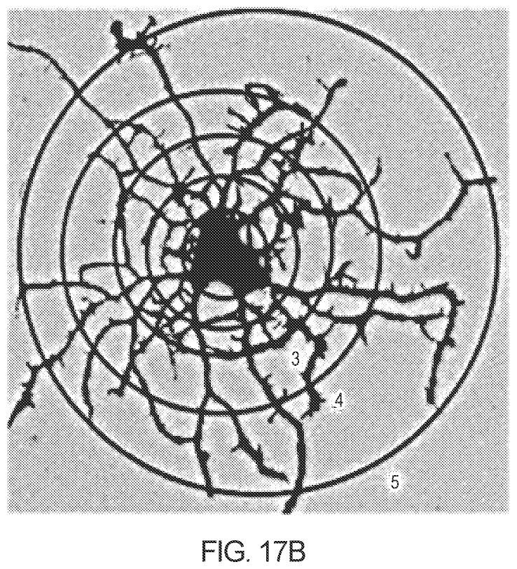

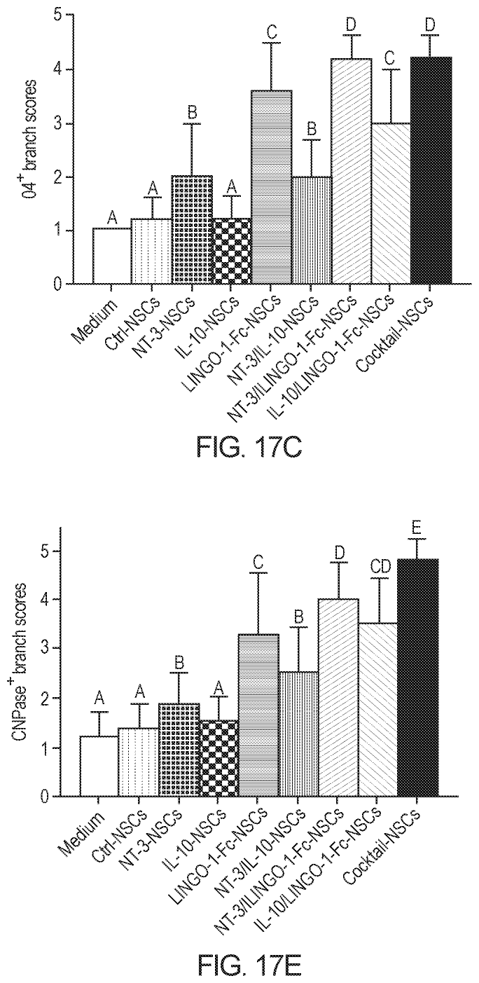

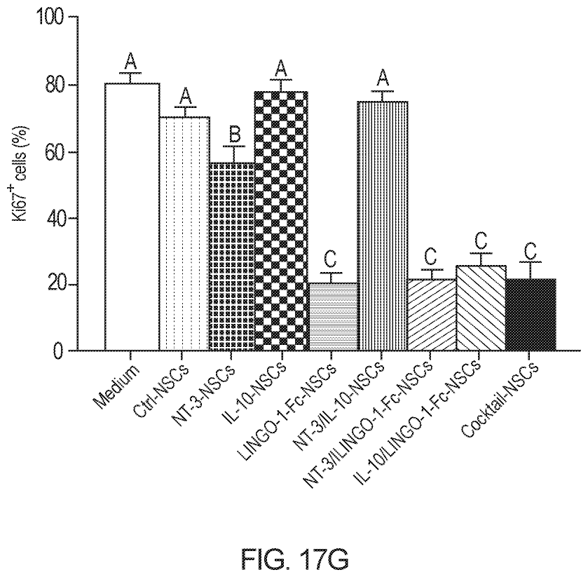

We also hypothesized that a combination of NT-3 and LINGO-1-Fc might enhance MBP synthesis [35, 36]. To test this, we used the transwell system to determine their effects on OPC differentiation in vitro. OPCs treated with a combination of LINGO-1-Fc- and NT-3-NSCs had dramatically greater branch range of pre-oligodendrocytes (O4+ cells) (FIG. 17a-c) and mature oligodendrocytes (CNPase+cells) with myelin sheaths (FIG. 17d, e). In the presence of NT-3/LINGO-1-Fc-NSCs and cocktail-NSCs, the CNPase+ oligodendrocytes exhibited complex membrane morphology compared with other groups (FIG. 17d), indicating that LINGO-1-Fc directly and strongly promoted OPC differentiation into mature oligodendrocytes and had the best effects in tandem with NT-3. A significantly decreased percentage of Ki67+ OPCS in cultures where NT-3 and LINGO-1-Fc were present (FIG. 17f, g) indicated an exit from cell cycle and the beginning of oligodendrocyte differentiation.

### DISCUSSION

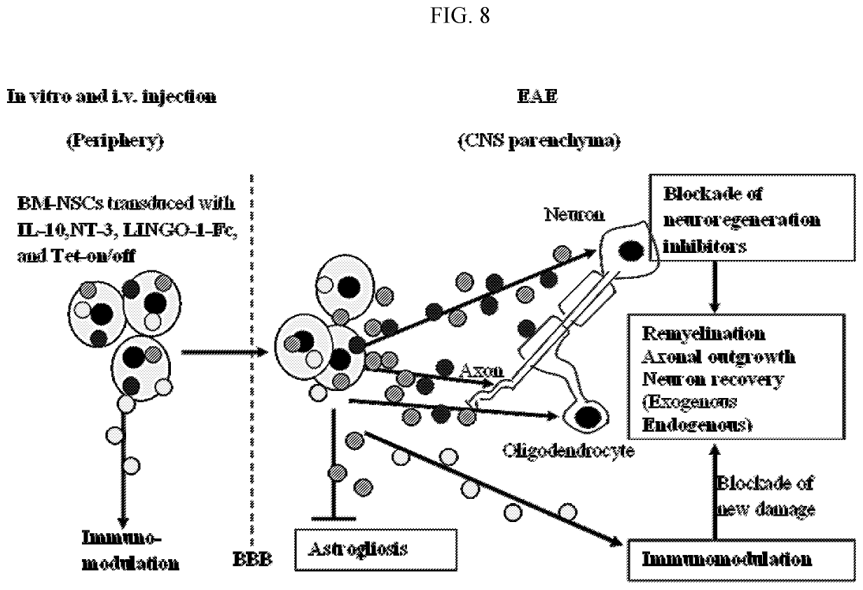

Treatment of chronic, progressive MS remains a major challenge due to the three major mechanisms that contribute to disease progression, including: 1) persistent CNS inflammation [7]; 2) loss of trophic support for both oligodendrocytes and neurons [8]; and 3) accumulation of CNS regeneration inhibitors [9]. While unmodified NSCs can suppress EAE when administered at the acute stage (e.g., day 22 p.i.) [6, 16], they failed to do so when administered at the chronic stage of disease (e.g., day 60 p.i.) as shown in the present study, most likely due to their weak anti-inflammatory capacity and low neurotrophin production [6, 16, 26]. To simultaneously target the three major pathogenic mechanisms underlying the failure of neuroregeneration at the chronic stage of EAE, here we have for the first time engineered NSCs expressing a therapeutic cocktail that are triply-effective (FIG. 8): 1) NT-3 supports NSC proliferation and for their differentiation into neurons and oligodendrocytes; 2) LINGO-1-Fc blocks the harmful effect of neuroregeneration inhibitors on OPCS/oligodendrocytes; and 3) the inhibitory effect of IL-10 on CNS inflammation and the inductive effect on M2 phenotype of microglia/macrophages., create a supportive microenvironment for neuroregeneration. Although transplanted NSCs can differentiate into neural cells, the majority of MBP-stained areas and neural cells were GFP-, indicating that promoting development of endogenous cells is the main mechanism of cocktail-induced remyelination and neuroregeneration [16, 21, 37].

An important feature of chronic EAE and MS is the loss of both oligodendrocytes and neurons in damaged CNS tissues [38]. An effective treatment for these diseases at the chronic stage would therefore be capable of inducing proliferation and differentiation of endogenous neural precursor cells, e.g., NSCs and OPCs, thus supplying sufficient neural cells for repair. NT-3 is an excellent candidate for this purpose, given its capacity to promote remyelination, axonal regeneration, and functional CNS recovery [39]. Compared to other neurotrophic factors, NT-3 has a significantly greater capacity to provide neuroprotection and reduce astrogliosis [40], a main cause of MS plaque formation [34]. NT-3 overexpression has been shown to enhance spinal cord injury recovery [41]. Our previous studies showed that NSCs transduced with NT-3 significantly enhanced neuroregeneration in acute EAE [26]. NT-3 acted on donor cells not only in an autocrine, but also in a paracrine fashion to enhance neuronal differentiation of both transplanted and endogenous cells, thus promoting neural repair [26]. Here, NT-3-NSCs significantly promoted NSC proliferation and differentiation towards neurons and oligodendrocytes, both of which are essential for neural repair in the chronic stage of EAE [42]. Although using NT-3 alone at the chronic stage was somewhat effective, in combination with other therapeutic molecules such as IL-10 and/or LINGO-1-Fc, its effect was significantly enhanced. These results indicate that, while immunomodulation and blocking neuroregeneration inhibitors are important, NT-3, together with other therapeutic factors, helps to achieve the maximal therapeutic effect in chronic EAE.

Another pathogenic mechanism underlying the failure of spontaneous remyelination in EAE and MS is the accumulation of neuroregeneration inhibitors, including Nogo, MAG and OMgp [9, 43]. These inhibitors are generated from debris of damaged CNS tissues, and they exert their effect on neural cells through the functional component of the NgR1/p75/LINGO-1 and NgR1/TAJ(TROY)/LINGO-1 receptor complexes that mediate inhibitory signals on axonal growth and oligodendrocyte differentiation [14]. LINGO-1 is a common, key factor in the above-mentioned complexes, and blocking LINGO-1 function using different approaches, including neutralizing antibodies, gene knockout, and LINGO-1-Fc administration, effectively promotes OPC maturation (FIG. 17a, d) and remyelination (FIG. 5a-d), and protects these cells from CNS inflammation- or chemical-induced damage [14, 44]. In the present study, we showed that among three transduced molecules, overexpression of LINGO-1-Fc most effectively enhanced MBP intensity in vivo and induced oligodendrocyte maturation in vitro. The importance of blocking LINGO-1 signaling in the inhibition of astrogliosis is supported by significantly reduced expression levels of GFAP, Vimentin, and N-cadherin in LPS-activated astrocytes in vitro. Furthermore, adding LINGO-1-Fc significantly enhanced the therapeutic effects of NSCs transduced with IL-10 and/or NT-3, clearly demonstrating the non-redundant role of LINGO-1-Fc in the cocktail.

Given that persistent CNS inflammation and microglia activation are considered the main cause of continuous neural tissue damage and EAE chronicity [45], treatment for neuroregeneration alone will be insufficient to ameliorate disease development, and it will need to be combined with an immunomodulation. Among immunomodulatory molecules used in EAE studies, IL-10, as the major product and common functional factor of regulatory T cells, tolerogenic DCs, and type 2 macrophages (M2), is considered highly potent in decreasing pro-inflammatory cytokine production [29, 46, 47]. While its systemic administration did not suppress EAE, IL-10 delivery by fibroblast cells into CNS inflamed foci effectively improved disease outcome [48]. We have shown that i.v. transplanted brain tissue-derived NSCs suppressed ongoing acute EAE and that their effects were significantly enhanced by transduction with IL-10 [6]. In the present study we observed that in acute EAE levels of pro-inflammatory cytokines were markedly lower in IL-10-NSC-treated mice than in sham-treated or NT-3/LINGO-1-Fc-NSC-treated EAE mice. Further, reduced pro-inflammatory cytokines and M1 response, together with enhanced M2 response, likely converted the CNS microenvironment in inflamed foci from hostile to supportive of neuroregeneration. Indeed, M2 cells are considered important for OPCs to mature into myelinating oligodendrocytes [30]. While IL-10 was a well-known anti-inflammatory molecular which ameliorate inflammation progressive in autoimmune disease, the clues from our results indicated IL-10 probably blockade demyelination by inhibited inflammatory cytokine expression and induced M2 macrophages/microglia response.

An ideal combination of multiple therapeutic reagents would enhance the effect of each of them. Interestingly, when IL-10 and NT-3 were combined, microglia activation was markedly inhibited compared with the group that was treated with IL-10 alone. Further, the combination of LINGO-1-Fc and NT-3 also markedly halted disease progression. Our in vitro data demonstrated that LINGO-1-Fc could significantly promote OPC differentiation into mature oligodendrocytes. However, we also found that NT-3 dramatically enhanced cell survival. A previous study showed that LINGO-1-Fc treatment promotes TrkB phosphorylation [14] and NT-3 combines with the Trk family receptor to activate its downstream pathway [39]. Treatment with NT-3 and LINGO-1-Fc may, therefore, enhance their effect to boost OPC differentiation and improve the treatment results of each of them compared with a single gene treatment.

Moreover, inflammation and demyelination are the two major elements of pathogenesis and exert their effects in parallel in chronic EAE. Our results show that IL-10 not only decreases pro-inflammatory cytokine production, but also dramatically inhibits microglial activation. Surprisingly, NT-3 had no effect on microglial activation, but when combined with IL-10 it appeared to enhance the suppressive capacity of the latter. This enhancement might be attributable to the upregulation of IL-10 production by NT-3 (FIG. 13e), although the exact mechanism is not clear. The superior effect of cocktail over single gene or combinations of any two of these genes indicates that these three therapeutic genes in the cocktail enhance the effects of one another in reversing CNS autoimmune damage.

The safety issue of NSC transplantation has been a concern for clinical application. For example, co-injection of allogeneic adult NSCs with pancreatic islets resulted in NSC-derived tumor formation [49] However, It has been found that NSCs, after transplantation, survive in the CNS for up to 15 months, without any sign of deleterious outcomes such as tumor formation, adverse immune responses or inappropriate anatomical accumulation [50]. Safety of lentiviral vector as a tool for gene therapy has been widely tested in clinical trials [51, 52]. We in another series of experiments observed that all cocktail-NSC-injected EAE mice survived up to 300 days p.i. without toxicity or tumor formation (data not shown). We, therefore, anticipate that the current system is an effective and safe method for the future therapy. Phase I and II clinical trials using NSCs as therapies are ongoing to test their safety and practicability [53].

Taken together, our data demonstrate that NSCs engineered to produce a “cocktail” of three therapeutic molecules effectively target the three major mechanisms underlying EAE chronicity and convert the hostile environment into one supportive of the neurodegenerative process, thus significantly promoting endogenous oligodendrocyte/neuron differentiation and axonal integrity. Further, in addition to being a delivery vehicle, NSCs have intrinsic capacity, albeit weak, for immunomodulation [21, 23], neural protection [54] and neural-repopulation [16, 19], functions that can be greatly enhanced by this therapeutic cocktail. Thus, this study provides a new approach to break the vicious cycle of inflammation, myelin damage and neuroregeneration blockade, and paves the way to a novel, inducible, and highly effective therapy for chronic CNS inflammatory demyelination, for which there is currently no effective therapy.

### METHODS

### Generation of NSCs and Cell Culture

NSCs were generated from bone marrow (BM) of the femurs of C57BL/6 mice (Jackson Laboratory, Bar Harbor, Me.), 6-8 weeks of age, as described previously [55]. Cells were plated on poly-D-lysine/laminin (Sigma-Aldrich, St. Louis, Mo.) coated 24-well plate and cultured in serum-free DMEM/F-12 (Invitrogen, Gaithersberg, Md.) supplemented with 2% B27, 20 ng/ml epidermal growth factor (EGF), and 20 ng/ml basic fibroblast growth factor (bFGF) along with antibiotics. Cells were plated at a density of 1×106 cells/well, and media changed every four days. After 2 weeks, a proportion of individual cells proliferated to form distinct neurosphere. After 3-4 weeks, the neurospheres were collected, dissociated to single cells by Accutase (Invitrogen), and replated at 1.0×105 cells/ml for the next passage. Expression of neural specific markers Nestin and Sox2 was determined by immunocytochemistry. Cells at 5th to 10th passages were used in the following in vitro and in vivo experiments.

Primary astrocytes, microglial cells and oligodendrocyte progenitor cells (OPCs) were isolated from newborn mouse brain, dissociated with Neural Tissue Dissociation Kit (Miltenyi Biotech Inc.) and purified with either anti-ASCA-2, anti-CD11b, or anti-A2B5 microbeads (Mitenyi Biotech Inc.) respectively. Astrocytes were cultured in DMEM/10% FBS cell culture medium, microglia in DMEM/10% FBS plus 5 ng/ml M-CSF (PeproTech, Rochy Hill, NJ), and OPCs in DMEM/F12 supplemented with 2% B27, 2 mM Glutamax, 20 ng/ml bFGF and 20 ng/ml PDGF-AA (Invitrogen).

### Construction of Vectors Inducibly Producing a Therapeutic Cocktail

To generate vectors inducibly expressing multiple therapeutic genes, we used the pLVX-EF1a-Tet3G and pLVX-TRE3G-IRES vectors (Clontech, Moutain View, Calif.). In brief, we cloned and inserted the NT-3 (SEQ ID No. 9) and IL-10 (SEQ ID No. 10) cDNA into multiple cloning sites (MCSs) of the pLVX-TRE3G-IRES-puro vector. Primer sequences are listed in Table 1. The puromycin gene was replaced by the hygromycin gene in the pLVX-TRE3G-IRES-puro vector, which was then named pLVX-TRE3G-IRES-hyg. Next, we inserted LINGO-1-Fc (SEQ ID No. 11) and copGFP cDNA into the MCS of pLVX-TRE3G-IRES-hyg. The Lenti-X HTX Packaging System (Clontech) was used to produce the lentivirus of Lenti-X Tet-On 3G, LV-TRE3 G-NT-3-IRES-IL-10, and LV-TRE3G-LINGO-1-Fc-IRES-copGFP (FIG. 10a).

To establish the complete Tet-On Advanced System, we performed sequential transduction with the lentiviruses into NSCs following the user's manual for the Lenti-X Tet-On 3G inducible expression system. NSCs were first transduced with only the Lenti-X Tet-On 3G, followed by selection with G418 (500 μg/ml). Resistant clones were then screened for Tet-On expression and tested for inducibility. A favorable Tet-On positive clone was then transduced with the LV-TRE3G-NT-3-IRES-IL-10 lentivirus. Doubly transduced cells were selected using puromycin (5 μg/ml), and the resulting puromycin-resistant clones were then screened for NT-3 and IL-10 inducibility. Afterwards, the neomycin and puromycin resistant clone was transduced with the LV-TRE3G-LINGO-1-Fc-IRES-copGFP lentivirus and, finally, triply-transduced cells were selected using hygromycin (500 μg/ml) and the resulting hygromycin-resistant clones screened for LINGO-1-Fc and copGFP inducibility. NSC-transduced with Lenti-X Tet-On 3G, LVX-TRE3G-IRES-puro and LVX-TRE3G-IRES-copGFP-hyg were used as control NSCs.

Proliferation and differentiation of cocktail-NSCs

The proliferation capacity of NSCs was tested by staining for Ki-67 in vitro. NSCs were seeded at a density of 2.0×104 cells/ml and incubated in NSC medium. After 24 hours, cells were fixed and stained using anti-Ki-67 antibody (Abcam, Cambridge, Mass.). The proliferation capacity was expressed as the percentage of Ki-67+ positive cells among GFP+ cells.

To evaluate the differentiation ability of engineered NSCs, dissociated single cells were plated on poly-D-lysine/laminin coated coverslip at a density of 2.0×104 cells/ml and cultured in specific NSC differentiation medium. In brief, for neuron differentiation, Neurobasal medium was supplemented with 2% B-27, 2 mM GlutaMax-I and 0.5 mM cAMP. For astrocyte differentiation, DMEM was supplemented with 1% N-2, 2 mM GlutaMax-I and 1% FBS. The oligodendrocyte differentiation medium requires Neurobasl medium supplemented with 2% B-27, 2 mM GlutaMax-I and 20 ng/ml T3. Over two weeks, NSCs in differentiation media changed morphology and developed markers of neurons, astrocytes and oligodendrocytes as determined by immunocytochemistry staining. To determine the number of cells expressing a specific antigen, five areas of each coverslip were examined, and the percentage of positive cells labeled for a specific neural marker in the total number of DAPI+cells was expressed as the mean value of specific neural differentiation.

### EAE Induction and Treatment

Female C57BL/6 mice, 7-8 weeks of age, were purchased from the Jackson Laboratory (Bar Harbor, ME). All experimental procedures and protocols were approved by the Institutional Animal Care and Use Committee of Thomas Jefferson University and were carried out in accordance with the approved institutional guidelines and regulations. Mice were immunized at 2 sites on the back with 200 μg of myelin oligodendrocyte glycoprotein peptide 35-55 (MOG35-55) (Genescript, Piscataway, N.J.) in 200 μl of emulsion containing 50% complete Freund's adjuvant with 5 mg/ml Mycobacterium tuberculosis H37Ra (Difco, Lawrence, Kan.). All mice were i.p. injected with 200 ng pertussis toxin (Sigma-Aldrich) in PBS on days 0 and 2 post immunization (p.i.). Clinical EAE was scored daily in a blind manner, according to a 0-5 scale as described previously [56]: 0, no clinical signs; 0.5, stiff tail; 1, limp tail; 1.5, limp tail and wadding gait; 2, paralysis of one limb; 2.5, paralysis of one limb and weakness of another limb; 3, complete paralysis of both hind limbs; 4, moribound; and 5, death.

NSCs transduced with different vectors were i.v. injected as single-cell suspension (1.0×106 cells in 150 μl PBS/each mouse) via the tail vein at the onset (day 10 p.i.) or chronic stage (day 60 p.i.) of disease. Upon cell injection, doxycycline (Dox; Sigma-Aldrich) was administered subcutaneously (s.c.) at a dose of 100 mg/kg/day [57]. Mice injected with PBS only served as control. The group using unmodified NSCs was omitted given that showed therapeutic effects similar to GFP-transduced NSCs (data not shown). In addition, given that injection of Dox alone did not affect the clinical course of EAE [26], a control group treated with Dox only was not included in the current study.

### Histological Analysis

Mice were sacrificed at day 28 (for acute EAE) or day 120 (for chronic EAE) p.i. and transcardially perfused with PBS. Brain and lumbar spinal cords were harvested for pathological assessment and spleen for immunological assessment. CNS tissues were cut into 7 μm sections, fixed with 4% paraformaldehyde and stained with hematoxylin and eosin (H&E) for assessment of inflammation and Luxol fast blue (LFB) for demyelination. Slides were assessed and scored in a blinded fashion for inflammation [58]:0, none; 1, a few inflammatory cells; 2, organization of perivascular infiltrates; and 3, abundant perivascular cuffing with extension into the adjacent tissue. For demyelination quantification, total white matter was manually outlined, and area (%) of demyelination was calculated by Image-Pro Plus software [59].

For immunohistochemistry, brain and spinal cord tissues were fixed using 4% paraformaldehyde for 1 day and then cryo-protected using 30% sucrose solution for 3 days. Fixed tissues were embedded in OCT compound (Tissue-Tek, Sakura Finetek, Japan) for frozen sections and then sectioned coronally at 12 μm. Transverse sections of brain and spinal cord were cut, and stained with different antibodies. Immunofluorescence controls were routinely generated by omitting primary antibodies. Results were visualized by fluorescent microscopy (Nikon Eclipse E600; Nikon, Melville, N.Y.) or confocal microscopy (Zeiss LSM 510; Carl Zeiss, Thornwood, N.Y.). For the quantifications of HE, FB, iNOS+Iba1+, Arg1+Iba1+, APP+, GFAP+ and MBP+, ten areas in the white matter at the lumbar spinal cord were selected (FIG. 11).

### Toludine Blue Staining and Analysis

For toluidine blue staining, spinal cords were dissected out and incubated in 2% paraformaldehyde plus 2.5% glutaraldehyde overnight. Fixed tissues were then immersed in 2% osmium tetroxide, dehydrated through a graded acetone series, and embedded into EPON following procedures described previously [44]. Semi-thin sections of 1 μm thickness were stained with 1% toluidine blue and images captured by light microscopy. A minimum of 10 micrographs per mouse were obtained, and 5 mice per group were evaluated. All axons in the area of ventral spinal cord were counted. To quantify myelinated axons, a line-sampling method was used according to the previous study [44]. G ratio, i.e., axon diameter divided by entire myelinated fiber diameter, was determined using ImageJ software [6, 60].

### Real-Time RT-PCR

Total RNA was extracted from cells or tissues using RNeasy® Plus Mini Kit (QIAGEN, Valencia, Calif.) according to the manufacturer's instructions. Reverse transcription was conducted using QuantiTect® Reverse Transcription Kit (QIAGEN). Real-time PCR was performed using the QuantiFast™ SYBR® Green PCR Kit (QIAGEN), and detection was performed using the ABI Prism® 7500 Sequence Detection System (Applied Biosystems, Foster City, Calif.). Gapdh was used as an internal control. Nucleotide sequences of the primers were based on published cDNA sequences.

### Mononuclear Cell (MNC) Preparation

Spleen was mechanically dissociated through a 70 μm cell strainer (Falcon, Tewksbury, Mass.) and incubated with red blood cell lysis buffer (Miltenyi) for 1 min. Harvested cells were washed with cold PBS before in vitro stimulation. To acquire CNS cells, spinal cords and brains were mechanically dissociated and incubated with Liberase (Roche, Nutley, N.J.) for 30 min, passed through a 70 μm cell strainer and washed with cold PBS. Cells were then fractionated on a 70/30% Percoll (Sigma-Aldrich) gradient by centrifugation at 2000 rpm for 20 min and MNCs were collected from the interface and washed with PBS.

### Cytokine Production by ELISA

Splenocytes at 1.0×106 cells/ml were cultured in triplicates in RPMI 1640 supplemented with 10% FBS in 24-well plates and stimulated with 25 μg/ml MOG35-55 for 72 hours. Supernatants were collected and assayed for IFN-γ, IL-17, GM-CSF, IL-5 and IL-10 by ELISA Kits (R&D System, Minneapolis, Minn.).

### Flow Cytometry Analysis

For surface-marker staining, cells were incubated with fluorochrome-conjugated Abs to CD4, CD8, CD11b, and CD11c (BD Biosciences, San Jose, Calif.) or isotype control Abs for 30 min on ice. For intracellular staining, splenocytes or CNS-infiltrating MNCs were stimulated with 25 μg/ml and 10 μg/ml MOG peptide for 72 hours or overnight, respectively, followed by stimulation with 50 ng/ml PMA and 500 ng/ml ionomycin in the presence of GolgiPlug for 5 hours. Cells were surface-stained with mAbs against CD4 and CD8. Cells were then washed, fixed, and permeabilized with FIX&PERM solution (Invitrogen), and intracellular cytokines were stained with Abs against IL-17, IFN-γ, or GM-CSF (BD Biosciences). Flow cytometric analysis was performed on FACSAria (BD Biosciences) and data were analyzed with FlowJo software (Treestar, Ashland, Oregon).

### Statistical Analysis

Statistical analyses were performed using GraphPad Prism 6 software (GraphPad, La Jolla, Calif.). Data are presented as mean±SD. When comparing multiple groups, data were analyzed by analysis of variance (ANOVA) with Tukey's multiple comparisons test. A significance criterion of p<0.05 was used for all statistical analysis.

### Therapeutic Treatments

As described herein, NSCs are generated that express several proteins at once that, when combined, provide for high efficacy for treatment of Multiple Sclerosis, and its mouse model. Accordingly, for human treatment, it may be appropriate to replace mouse cDNA for each of the proteins with human proteins. Accordingly, homology to the sequences provided herein is at least 50%, at least 60%, at least 70%, at least 80%, at least 90%, at least 95%, and at least 99%. Those of ordinary skill in the art will recognize that we can exchange the mouse cDNA for human cDNA and use a human stem cell from bone marrow as described herein.

Because of the homology between the mouse model and the human model, the EAE model provides for a highly analogous model for confirming the efficacy of the treatments provided herein.

TABLE 1
Primers used for pLV-TRE3G-NT-3-IRES-IL-10
and pLV-TRE3G-LINGO-1-Fc-IRES-copGFP vector
construction

| Primer name | SEQ ID NOS. 1-8 (5′-3′) |
|---|---|
| NT-3 Fwd | ACT ACG CGT ATG TCC ATC TTG TTT TAT GT |
| NT-3 Rev | CTT GAA TTC TCA TGT TCT TCC AAT TTT |
| IL-10 Fwd | ACT GGA TCC ATG CCT GGC TCA GCA CTG CT |
| IL-10 Rev | CTT GCG GCC GCT TAG CTT TTC ATT TTG |
| | ATC A |
| LINGO-1-Fc | ACT GGA TCC CCA CCA TGT ACA GGA TGC AA |
| Fwd | |
| LINGO-1-Fc | CTT GCG GCC GCC TAT TTA CCC GGA GAC AGG |
| Rev | GAG A |
| copGFP Fwd | ACT ACG CGT ATG GAG AGC GAC GAG AGC GG |
| copGFP Rev | CTT GGA TCC TTA GCG AGA TCC GGT GGA GC |

### REFERENCES

1. Rao, P, and Segal, B M (2012). Experimental autoimmune encephalomyelitis. Methods in molecular biology (Clifton, N.J.) 900: 363-380.
2. Baxi, E G, and DeBruin, J (2015). Transfer of myelin-reactive th17 cells impairs endogenous remyelination in the central nervous system of cuprizone-fed mice 35: 8626-8639.
3. Raphael, I, Nalawade, S, Eagar, T N, and Forsthuber, T G (2015). T cell subsets and their signature cytokines in autoimmune and inflammatory diseases. Cytokine 74: 5-17.
4. Mascanfroni, I D, Yeste, A, Vieira, S M, Burns, E J, Patel, B, Sloma, I, et al. (2013). IL-27 acts on DCs to suppress the T cell response and autoimmunity by inducing expression of the immunoregulatory molecule CD39. Nature immunology 14: 1054-1063.
5. Lukens, J R, Gurung, P, Shaw, P J, Barr, M J, Zaki, M H, Brown, S A, et al. (2015). The NLRP12 Sensor Negatively Regulates Autoinflammatory Disease by Modulating Interleukin-4 Production in T Cells. Immunity 42: 654-664.
6. Yang, J, Jiang, Z, Fitzgerald, D C, Ma, C, Yu, S, Li, H, et al. (2009). Adult neural stem cells expressing IL-10 confer potent immunomodulation and remyelination in experimental autoimmune encephalitis. The Journal of clinical investigation 119: 3678-3691.
7. Pluchino, S, Muzio, L, Imitola, J, Deleidi, M, Alfaro-Cervello, C, Salani, G, et al. (2008). Persistent inflammation alters the function of the endogenous brain stem cell compartment. Brain : a journal of neurology 131: 2564-2578.
8. Franklin, R J, and Ffrench-Constant, C (2008). Remyelination in the CNS: from biology to therapy. Nature reviews Neuroscience 9: 839-855.
9. Yang, Y, Liu, Y, Wei, P, Peng, H, Winger, R, Hussain, R Z, et al. (2010). Silencing Nogo-A promotes functional recovery in demyelinating disease. Annals of neurology 67: 498-507.
10. Rasmussen, S, Imitola, J, Ayuso-Sacido, A, Wang, Y, Starossom, SC, Kivisakk, P, et al. (2011). Reversible neural stem cell niche dysfunction in a model of multiple sclerosis. Annals of neurology 69: 878-891.
11. Popescu, B F, and Lucchinetti, C F (2012). Pathology of demyelinating diseases. Annual review of pathology 7: 185-217.
12. Ransohoff, R M, Hafler, D A, and Lucchinetti, C F (2015). Multiple sclerosis-a quiet revolution. Nature reviews Neurology 11: 134-142.
13. Acosta, C M, Cortes, C, MacPhee, H, and Namaka, M P (2013). Exploring the role of nerve growth factor in multiple sclerosis: implications in myelin repair. CNS & neurological disorders drug targets 12: 1242-1256.
14. Mi, S, Pepinsky, R B, and Cadavid, D (2013). Blocking LINGO-1 as a therapy to promote CNS repair: from concept to the clinic. CNS drugs 27: 493-503.
15. Ben-Hur, T, Einstein, O, Mizrachi-Kol, R, Ben-Menachem, O, Reinhartz, E, Karussis, D, et al. (2003). Transplanted multipotential neural precursor cells migrate into the inflamed white matter in response to experimental autoimmune encephalomyelitis. Glia 41: 73-80.
16. Pluchino, S, Quattrini, A, Brambilla, E, Gritti, A, Salani, G, Dina, G, et al. (2003). Injection of adult neurospheres induces recovery in a chronic model of multiple sclerosis. Nature 422: 688-694.
17. Pluchino, S, and Cossetti, C (2013). How stem cells speak with host immune cells in inflammatory brain diseases. Glia 61: 1379-1401.
18. Giusto, E, Donega, M, Cossetti, C, and Pluchino, S (2014). Neuro-immune interactions of neural stem cell transplants: from animal disease models to human trials. Experimental neurology 260: 19-32.
19. Laterza, C, Merlini, A, De Feo, D, Ruffini, F, Menon, R, Onorati, M, et al. (2013). iPSC-derived neural precursors exert a neuroprotective role in immune-mediated demyelination via the secretion of LIF. Nature communications 4: 2597.
20. De Feo, D, Merlini, A, Laterza, C, and Martino, G (2012). Neural stem cell transplantation in central nervous system disorders: from cell replacement to neuroprotection. Current opinion in neurology 25: 322-333.
21. Pluchino, S, Zanotti, L, Rossi, B, Brambilla, E, Ottoboni, L, Salani, G, et al. (2005). Neurosphere-derived multipotent precursors promote neuroprotection by an immunomodulatory mechanism. Nature 436: 266-271.
22. Yang, J, Yan, Y, Ciric, B, Yu, S, Guan, Y, Xu, H, et al. (2010). Evaluation of bone marrow- and brain-derived neural stem cells in therapy of central nervous system autoimmunity. The American journal of pathology 177: 1989-2001.
23. Einstein, O, Karussis, D, Grigoriadis, N, Mizrachi-Kol, R, Reinhartz, E, Abramsky, O, et al. (2003). Intraventricular transplantation of neural precursor cell spheres attenuates acute experimental allergic encephalomyelitis. Molecular and cellular neurosciences 24: 1074-1082.
24. Mi, S, Lee, X, Shao, Z, Thill, G, Ji, B, Relton, J, et al. (2004). LINGO-1 is a component of the Nogo-66 receptor/p75 signaling complex. Nat Neurosci 7: 221-228.
25. Serwer, L, Hashizume, R, Ozawa, T, and James, CD (2010). Systemic and local drug delivery for treating diseases of the central nervous system in rodent models. J Vis Exp.
26. Yang, J, Yan, Y, Xia, Y, Kang, T, Li, X, Ciric, B, et al. (2014). Neurotrophin 3 transduction augments remyelinating and immunomodulatory capacity of neural stem cells. Molecular therapy : the journal of the American Society of Gene Therapy 22: 440-450.
27. El-Behi, M, Ciric, B, Dai, H, Yan, Y, Cullimore, M, Safavi, F, et al. (2011). The encephalitogenicity of T(H)17 cells is dependent on IL-1- and IL-23-induced production of the cytokine GM-CSF. Nature immunology 12: 568-575.
28. Bannerman, P G, Hahn, A, Ramirez, S, Morley, M, Bonnemann, C, Yu, S, et al. (2005). Motor neuron pathology in experimental autoimmune encephalomyelitis: studies in THY1-YFP transgenic mice. Brain : a journal of neurology 128: 1877-1886.
29. Zhang, X M, Lund, H, Mia, S, Parsa, R, and Harris, R A (2014). Adoptive transfer of cytokine-induced immunomodulatory adult microglia attenuates experimental autoimmune encephalomyelitis in DBA/1 mice. Glia 62: 804-817.
30. Miron, V E, Boyd, A, Zhao, J W, Yuen, T J, Ruckh, J M, Shadrach, J L, et al. (2013). M2 microglia and macrophages drive oligodendrocyte differentiation during CNS remyelination. Nature neuroscience 16: 1211-1218.
31. Zawadzka, M, and Franklin, R J (2007). Myelin regeneration in demyelinating disorders: new developments in biology and clinical pathology. Current opinion in neurology 20: 294-298.
32. Bando, Y, Nomura, T, Bochimoto, H, Murakami, K, Tanaka, T, Watanabe, T, et al. (2015). Abnormal morphology of myelin and axon pathology in murine models of multiple sclerosis. Neurochemistry international 81: 16-27.
33. Papadopoulos, D, Pham-Dinh, D, and Reynolds, R (2006). Axon loss is responsible for chronic neurological deficit following inflammatory demyelination in the rat. Experimental neurology 197: 373-385.
34. Frohman, E M, Racke, M K, and Raine, C S (2006). Multiple sclerosis--the plaque and its pathogenesis. The New England journal of medicine 354: 942-955.
35. Fu, Q L, Li, X, Yip, H K, Shao, Z, Wu, W, Mi, S, et al. (2009). Combined effect of brain-derived neurotrophic factor and LINGO-1 fusion protein on long-term survival of retinal ganglion cells in chronic glaucoma. Neuroscience 162: 375-382.
36. Tauszig-Delamasure, S, and Bouzas-Rodriguez, J (2011). Targeting neurotrophin-3 and its dependence receptor tyrosine kinase receptor C: a new antitumoral strategy. Expert opinion on therapeutic targets 15: 847-858.
37. Einstein, O, Fainstein, N, Vaknin, I, Mizrachi-Kol, R, Reihartz, E, Grigoriadis, N, et al. (2007). Neural precursors attenuate autoimmune encephalomyelitis by peripheral immunosuppression. Annals of neurology 61: 209-218.
38. Tully, M, and Shi, R (2013). New insights in the pathogenesis of multiple sclerosis--role of acrolein in neuronal and myelin damage. International journal of molecular sciences 14: 20037-20047.
39. Khan, N, and Smith, M T (2015). Neurotrophins and Neuropathic Pain: Role in Pathobiology. Molecules (Basel, Switzerland) 20: 10657-10688.
40. Girard, C, Bemelmans, A P, Dufour, N, Mallet, J, Bachelin, C, Nait-Oumesmar, B, et al. (2005). Grafts of brain-derived neurotrophic factor and neurotrophin 3-transduced primate Schwann cells lead to functional recovery of the demyelinated mouse spinal cord. The Journal of neuroscience : the official journal of the Society for Neuroscience 25: 7924-7933.
41. Hou, S, Nicholson, L, van Niekerk, E, Motsch, M, and Blesch, A (2012). Dependence of regenerated sensory axons on continuous neurotrophin-3 delivery. The Journal of neuroscience: the official journal of the Society for Neuroscience 32: 13206-13220.
42. Bai, L, Lennon, D P, Caplan, A I, DeChant, A, Hecker, J, Kranso, J, et al. (2012). Hepatocyte growth factor mediates mesenchymal stem cell-induced recovery in multiple sclerosis models. Nat Neurosci 15: 862-870.
43. Geoffroy, C G, and Zheng, B (2014). Myelin-associated inhibitors in axonal growth after CNS injury. Current opinion in neurobiology 27: 31-38.
44. Mi, S, Hu, B, Hahm, K, Luo, Y, Kam Hui, E S, Yuan, Q, et al. (2007). LINGO-1 antagonist promotes spinal cord remyelination and axonal integrity in MOG-induced experimental autoimmune encephalomyelitis. Nature medicine 13: 1228-1233.
45. Starossom, S C, Imitola, J, Wang, Y, Cao, L, and Khoury, S J (2011). Subventricular zone microglia transcriptional networks. Brain, behavior, and immunity 25: 991-999.
46. Kleinewietfeld, M, and Hafler, D A (2014). Regulatory T cells in autoimmune neuroinflammation. Immunological reviews 259: 231-244.
47. Xie, Z X, Zhang, H L, Wu, X J, Zhu, J, Ma, D H, and Jin, T (2015). Role of the immunogenic and tolerogenic subsets of dendritic cells in multiple sclerosis. Mediators of inflammation 2015: 513295.
48. Payne, N L, Sun, G, McDonald, C, Moussa, L, Emerson-Webber, A, Loisel-Meyer, S, et al. (2013). Human adipose-derived mesenchymal stem cells engineered to secrete IL-10 inhibit APC function and limit CNS autoimmunity. Brain, behavior, and immunity 30: 103-114.
49. Melzi, R, Antonioli, B, Mercalli, A, Battaglia, M, Valle, A, Pluchino, S, et al. (2010). Co-graft of allogeneic immune regulatory neural stem cells (NPC) and pancreatic islets mediates tolerance, while inducing NPC-derived tumors in mice. PloS one 5: e10357.
50. Lepore, A C, Neuhuber, B, Connors, T M, Han, SS, Liu, Y, Daniels, M P, et al. (2006). Long-term fate of neural precursor cells following transplantation into developing and adult CNS. Neuroscience 142: 287-304.
51. Hacein-Bey Abina, S, Gaspar, H B, Blondeau, J, Caccavelli, L, Charrier, S, Buckland, K, et al. (2015). Outcomes following gene therapy in patients with severe Wiskott-Aldrich syndrome. Jama 313: 1550-1563.
52. Oldham, R A, Berinstein, E M, and Medin, J A (2015). Lentiviral vectors in cancer immunotherapy. Immunotherapy 7: 271-284.
53. Casarosa, S, Bozzi, Y, and Conti, L (2014). Neural stem cells: ready for therapeutic applications? Molecular and cellular therapies 2: 31.
54. Aharonowiz, M, Einstein, O, Fainstein, N, Lassmann, H, Reubinoff, B, and Ben-Hur, T (2008). Neuroprotective effect of transplanted human embryonic stem cell-derived neural precursors in an animal model of multiple sclerosis. PloS one 3: e3145.
55. Yuan, X, Hu, J, Belladonna, M L, Black, K L, and Yu, J S (2006). Interleukin-23-expressing bone marrow-derived neural stem-like cells exhibit antitumor activity against intracranial glioma. Cancer research 66: 2630-2638.
56. Kataoka, H, Sugahara, K, Shimano, K, Teshima, K, Koyama, M, Fukunari, A, et al. (2005). FTY720, sphingosine 1-phosphate receptor modulator, ameliorates experimental autoimmune encephalomyelitis by inhibition of T cell infiltration. Cellular & molecular immunology 2: 439-448.

057. Liang, Y, Agren, L, Lyczek, A, Walczak, P, and Bulte, J W (2013). Neural progenitor cell survival in mouse brain can be improved by co-transplantation of helper cells expressing bFGF under doxycycline control. Experimental neurology 247: 73-79.
58. Yang, J, Yan, Y, Ma, CG, Kang, T, Zhang, N, Gran, B, et al. (2012). Accelerated and enhanced effect of CCR5-transduced bone marrow neural stem cells on autoimmune encephalomyelitis. Acta neuropathologica 124: 491-503.
59. Liu, C Y, Guo, S D, Yu, J Z, Li, Y H, Zhang, H, Feng, L, et al. (2015). Fasudil mediates cell therapy of EAE by immunomodulating encephalomyelitic T cells and macrophages. European journal of immunology 45: 142-152.
60. Cartoni, R, Arnaud, E, Medard, J J, Poirot, O, Courvoisier, D S, Chrast, R, et al. (2010). Expression of mitofusin 2(R94Q) in a transgenic mouse leads to Charcot-Marie-Tooth neuropathy type 2A. Brain : a journal of neurology 133: 1460-1469.

What is claimed is:

1. A therapeutic for treatment of multiple sclerosis comprising neural stem cells (NSCs) engineered with a vector to insert cDNA corresponding to neurotrophin 3 (NT-3), IL-10, and LINGO-1, wherein said NSC to simultaneously produce a cocktail containing IL-10, neurotrophin3, and soluble LINGO-1 protein.

2. The therapeutic of claim 1, wherein said NSC comprises a lenti virus vector inserting the cDNA sequence corresponding to NT-3, IL-10, and LINGO-1.

3. The therapeutic of claim 1 or 2, wherein said NSC comprises SEQ ID Nos. 9, 10, and 11.

4. The therapeutic of claims 1-3, suitable to inhibit pro-inflammatory M1 phenotype of microglia and switch it to M2 phenotype.

5. A method of treating multiple sclerosis comprising administering to a patient an effective dose of an engineered NSC producing a cocktail containing IL-10, neurotrophin3, and soluble LINGO-1 protein.

6. The method of claim 5, wherein said NSC comprises a vector inserting a sequence corresponding to NT-3, IL-10, and LINGO-1.

7. The method of claim 5, wherein said NSC comprises cDNA comprising SEQ ID Nos. 9, 10, and 11.

8. The therapeutic of claim 1, wherein said method inhibits pro-inflammatory M1 phenotype of microglia and switches it to M2 phenotype.

9. A method of transgenically modifying an NSC cell comprising: inserting NT-3, IL-10, and LINGO-1-Fc genes individually into separate vectors and transduced them into cells sequentially, wherein said transgenic cell is thereafter comprises genes corresponding to NT-3, IL-10, and LINGO-1, and wherein said transgenically modified NSC cells is capable of expressing NT-3, IL-10, and LINGO-1.

10. The method of claim 9, comprising generating a vector comprising NT-3 cDNA having primers corresponding to SEQ ID Nos 1, and 2.

11. The method of claim 10, wherein the NT-3 cDNA comprises SEQ ID No. 9.

12. The method of claim 9, comprising generating a vector comprising IL-10 cDNA having primers corresponding to SEQ ID Nos. 3, and 4.

13. The method of claim 12, wherein the IL-10 cDNA comprises SEQ ID No. 10.

14. The method of claim 9, comprising generating a vector comprising LINGO-1-Fc cDNA having primers corresponding to SEQ ID Nos. 5, and 6.

15. The method of claim 14, wherein the LINGO-1-Fc cDNA comprises SEQ ID No. 11.

16. The method of claim 9, comprising generating a vector comprising copGFP having primers corresponding to SEQ ID Nos. 7, and 8.

17. The method of claim 9, comprising sequentially inserting each of NT-3, IL-10, and LINGO-1-Fc into multiple cloning sites of at least one vector.

18. The method of claim 14, wherein said at least one vector is pLVX-EF1a-Tet3G.

19. The method of claim 14, wherein said at least one vector is pLVS-TRE3G-IRES.

20. The method of claim 14, wherein said at least one vector is a first and second vector comprising pLVX-EF1a and pLVX-TRE3G-IRES.

21. THe method of claim 20, wherein said first vector comprises SEQ ID Nos. 9 and 10, and said second vector comprises SEQ ID no. 11.

22. A method of producing a transgenic neural stem cell inducibly expressing multiple therapeutic genes comprising: cloning and inserting the NT-3 (SEQ ID No. 9) and IL-10 (SEQ ID No. 10) cDNA into multiple cloning sites (MCSs) of the pLVX-TRE3G-IRES-puroycin vector; said insertions are completed with primers comprising SEQ ID Nos. 1-4; replacing the puromycin gene with a hygromycin gene in the pLVX-TRE3G-IRES-puro vector, and inserting LINGO-1-Fc (SEQ ID No. 11) and into pLVX-TRE3G-IRES-puro vector after replacement of the puromycin gene with the hygromycin gene

23. The method of claim 22, wherein copGFP cDNA is also inserted into the MCS of pLVX-TRE3G-IRES-puro for insertion into the cell.

24. The method of claim 22, wherein the vectors are produced with a lentivirus.

25. The method of claim 24, wherein the lentivirus is a Lenti-X HTX Packaging System (Clontech) was used to produce the lentivirus of Lenti-X Tet-On 3G, LV-TRE3G-NT-3-IRES-IL-10, and LV-TRE3G-LINGO-1-Fc-IRES-copGFP.

26. A transgenic neural stem cell (NSC) engineered with a vector to insert cDNA corresponding to neurotrophin 3 (NT-3), IL-10, and LINGO-1, wherein said NSC to simultaneously produce a cocktail containing IL-10, neurotrophin3, and soluble LINGO-1 protein.

27. The therapeutic of claim 1, wherein said NSC comprises a lenti virus vector inserting the cDNA sequence corresponding to NT-3, IL-10, and LINGO-1.

28. The therapeutic of claim 26 or 27, wherein said NSC comprises SEQ ID Nos. 9, 10, and 11.

29. The therapeutic of claims 26-28, wherein SEQ IS Nos. 9, 10, and 11 are inserted using primers corresponding to SEQ ID Nos. 1-6.

30. The therapeutic of claims 26-29, suitable to inhibit pro-inflammatory M1 phenotype of microglia and switch it to M2 phenotype.

31. The therapeutic of any one of claims 1-30 comprising a 90% conserved sequence with SEQ ID Nos 9, 10, and 11.

32. Use of the therapeutic of claim 1-4 or 26-30 for treatment of EAE.

33. Use of the therapeutic of claim 1-4 or 26-30 for expressing a therapeutic cocktail within a patient, wherein said therapeutic cocktail comprises NT-3, IL-10, and LINGO-1-Fc.

34. A method of treating chornic stage EAE comprising administering to said patient a neural stem cell modified to express NT-3, IL-10 and LINGO-1-Fc.

35. The method of claim 33, wherein said neural stem cell is any one of claim 1-4 or 26-30.

36. A method of treating multiple sclerosis comprising administering to said patient a transgenic neural stem cell expressing NT-3, IL-10, and LINGO-1-Fc.

37. The method of claim 36 comprising sequences having 70% homology or greater to SEQ ID Nos. 9, 10, and 11.
